(19) United States
(12) Patent Application Publication
(10) Pub. No.: US 2021/0316068 A1
(43) Pub. Date: Oct. 14, 2021

(54) INFUSION PUMP AND INFUSION PUMP OPERATIONS

(71) Applicant: Takeda Pharmaceutical Company Limited, Osaka (JP)

(72) Inventors: Angela Teresa Muriset, Hoffman Estates, IL (US); Thomas Benjamin Roush, Niles, IL (US); Madeleine Clare Gibson, Madison, WI (US); Andrew Scott Argersinger, Bristol, WI (US)

(73) Assignee: Takeda Pharmaceutical Company Limited, Osaka (JP)

(21) Appl. No.: 17/282,290

(22) PCT Filed: Sep. 24, 2019
(86) PCT No.: PCT/US2019/052579

§ 371 Date: Apr. 1, 2021

Related U.S. Application Data
(60) Provisional application No. 62/740,592, filed on Oct. 3, 2018.

(51) Int. Cl. A61M 5/172; G16H 20/17; G16H 40/63; G16H 40/40

(52) U.S. Cl. CPC A61M 5/172; G16H 20/17; G16H 40/63; A61M 2005/14208; A61M 2205/502; A61M 2205/6009; G16H 40/40

(57) ABSTRACT

According to various aspects of the present application, embodiments of an infusion pump for administering medication to a patient are provided. According to one embodiment, the infusion pump automatically determines time intervals and associated rates of infusion of the medication, and automatically controls a pump engine to infuse the medication for the determined time intervals at the associated rates of infusion. According to another embodiment, the infusion pump determines whether an infusion set is configured to infuse medication into a patient at one site or at two sites. The infusion pump then controls the pump engine based on the determined configuration of the infusion set. According to another embodiment, the infusion pump automatically primes the infusion set for infusion of medication into a patient. According to another embodiment, the infusion pump automatically determines whether one or more needles have been placed on the patient correctly.

### CROSS-REFERENCE TO RELATED APPLICATIONS

This application claims the benefit under 35 U.S.C. § 119(e) of U.S. Provisional Application 62/740,592, filed on Oct. 3, 2018, which is incorporated herein by reference in its entirety.

### FIELD OF THE DISCLOSURE

The present disclosure relates to infusion pumps and associated techniques for automated infusion of medication into a patient.

### BACKGROUND

Medicinal fluids may be administered to patients using a medical infusion pump. A medical professional may operate the medical infusion pump, non-professional caregivers, or patients may use the infusion pump to administer medication to themselves.

Conventional infusion pumps can be complex to operate, as set-up of such infusion pumps may require the connection of specific infusion sets, syringes, and containers of medicinal fluid. Accordingly, conventional infusion pumps lack a streamlined procedure for performing specific administration processes. The inventors have recognized the need for an infusion pump that simplifies administration of medicinal fluid to a patient.

### SUMMARY OF THE DISCLOSURE

According to various aspects of the present application, various embodiments of an infusion pump for administering medication to a patient are provided. The inventors have developed various embodiments of an infusion pump that address various challenges with use of an infusion pump by a patient, and shortcomings in conventional infusion pumps. According to one embodiment, (1) the infusion pump receives user-specified time interval durations and automatically determines associated rates of infusion of the medication for each time interval, and (2) automatically controls a pump engine to infuse the medication for the time intervals at the associated rates of infusion. The infusion pump may determine the infusion rates based on prescribing information for the medication. According to another embodiment, the infusion pump determines whether an infusion set coupled to the infusion pump is configured to infuse medication into a patient at one infusion site or at two infusion sites. The infusion pump then controls the pump engine based on the determined infusion set type. According to another embodiment, the infusion pump aids a user in priming an infusion set. According to another embodiment, the infusion pump aids the user in performing a needle placement check by pulling back fluid so that the user can determine whether one or more needles have been inserted into the patient correctly.

### BRIEF DESCRIPTION OF DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 shows a perspective view of an embodiment of an infusion pump according to an aspect of the present application.

FIG. 2 shows a perspective view of the infusion pump of FIG. 1 with a compartment lid removed according to an aspect of the present application.

FIG. 3 shows an embodiment of a process for infusing medication into a patient by an infusion pump according to an aspect of the present application.

FIG. 4 shows an embodiment of a process for generating a medication infusion program by an infusion pump according to an aspect of the present application.

FIGS. 5A-5E show embodiments of user interface screens for creating a medication infusion program by an infusion pump according to an aspect of the present application.

FIG. 6 shows an example of data that may be used by an infusion pump for creating a medication infusion program according to an aspect of the present application.

FIG. 7 shows an embodiment of a process for configuring an infusion pump according to a determined type of infusion set received by the infusion pump according to an aspect of the present application.

FIGS. 8A-8D show embodiments of user interface screens generated by an infusion pump for determining a type of infusion set received by the infusion pump according to an aspect of the present application.

FIG. 9 shows an embodiment of a process for priming an infusion set using an infusion pump according to an aspect of the present application.

FIGS. 10A-10C show embodiments of user interface screens generated by an infusion pump for priming an infusion set according to an aspect of the present application.

FIG. 11 shows an embodiment of a process for priming an infusion set for infusion of multiple medications according to an aspect of the present application.

FIG. 12 shows an embodiment of a process for an infusion pump to check whether a needle has been placed correctly according to an aspect of the present application.

FIGS. 13A-13E show embodiments of user interface screens generated by an infusion pump for checking whether a needle has been placed correctly according to an aspect of the present application.

FIGS. 14A-14B show front and top views of an embodiment of a pump engine and infusion set sensor assembly according to an aspect of the present application.

FIG. 15 shows a perspective cross-sectional view of an embodiment of a pump engine shown in FIGS. 14A-14B according to an aspect of the present application.

FIG. 16 shows an embodiment of a pump engine disposed in an infusion pump according to an aspect of the present application.

FIG. 17 shows a schematic diagram of an infusion set according to an aspect of the present application.

FIG. 18 shows an embodiment of a computer system according an aspect of the present application.

### DETAILED DESCRIPTION

The inventors have recognized that conventional infusion pumps used for infusion of medication into a patient require the user (which may be the patient, a healthcare provider, or a non-professional caregiver) to manually set and modify rates of infusion of the medication. A prescription of the medication may specify that the medication is to be infused over a sequence of time intervals at varying rates of infusion. To administer the medication as specified by the prescription, the user may be required to calculate and/or look up particular infusion time intervals and their associated rates of infusion. In some cases the user must monitor time during infusion, and manually adjust rates of infusion to infuse the medication according to the prescription. In some cases, a user manually pre-programs an infusion pump, manually entering time interval durations and associated infusion rates. It may be difficult for a self-administering patient to adjust rates of infusion while infusing medication and/or to calculate the proper infusion rate to use. As a result, use of conventional infusion pumps for treatment of a patient may be time consuming and/or inconvenient.

Described herein are pump operations that facilitate ease of infusion for a user by providing automated processes or otherwise reducing manual steps needed to perform an infusion.

Some embodiments of the present application provide an improved infusion pump for administering medication to a patient that receives a user-specified time interval duration and automatically determines the proper infusion rate for each time interval based on the user's characteristics and the number of times the patient has received the infusion in the past. In some embodiments, the time interval duration is used to create a ramp up delivery profile in which the infusion pump increases the infusion rate after each time interval duration has been reached. In other words, the infusion pump infuses at a first infusion rate for a length of time equal to the time interval duration, and then ramps up to a second, increased infusion rate for the same length of time, and then ramps up to a third, increased infusion rate for the same length of time. As an example, if a user selects a time interval duration of 5 minutes, the infusion pump will ramp up the infusion rate every 5 minutes.

In some embodiments, the infusion pump will ramp up the infusion rate for a set number of time intervals, and then remain at a single infusion rate for the rest of the infusion. In some embodiments, the user can set the total number of desired time intervals. In some embodiments, the total number of time intervals is pre-set and a user cannot change the number. For example, the infusion pump may be pre-set to infusion at four ramp-up intervals, and the user can set the time duration for the intervals, but after the four intervals have been completed, the infusion pump may then infuse at a certain rate for the remainder of the infusion. This last infusion segment may be longer, equal to, or shorter in duration than the ramp-up time intervals.

In one illustrative embodiment, a user may enter an indication of the patient's weight, a dosage of the medication that the patient is to receive, how many times that patient has received the infusion in the past, and the user's desired time interval length for the ramp-up segment of the infusion. The infusion pump may then determine rates at which to infuse the medication during each time interval based on the indication of weight, dosage, and infusion history. In some embodiments, a user may enter an indication of a desired infusion interval duration, i.e. how long the infusion pump should infuse at a first rate before increasing to a higher rate during a ramp up period. The infusion pump may then automatically determine how many intervals should be used and an appropriate flow rate for each interval. The infusion pump eliminates the need for users to make determinations of rates at which to infuse the medication and eliminates the need for users to manually set or adjust infusion rates. Thus the infusion pump may provide a convenient way to deliver a medication.

In some embodiments, the infusion pump and pump operations described herein are used for subcutaneous infusion.

An infusion set used by the infusion pump to infuse medication into a patient may be configured to infuse medication at one site or multiple sites (e.g., two, or three) on the patient's body. For example, medication may be infused (1) into one site on the patient's body via a single needle, or (2) into multiple sites on the patient's body via multiple needles. Infusion of medication into the patient may vary based on the number of sites through which the infusion set is configured to infuse the medication. For example, times and rates of infusion may be different when infusing via one site compared to infusing via two sites.

The inventors have recognized that conventional infusion pumps are unable to determine whether the infusion pump is coupled to an infusion set that is configured to infuse medication at one site, two sites, or any number of sites. As a result, conventional infusion pumps are unable to adjust operation of the infusion pump based on the type of infusion set that is coupled to the infusion pump (e.g., whether the infusion set has a single needle for infusion into one site, or multiple needles for infusion into multiple sites.) Additionally, in some situations, a user must calculate infusion rates required for a particular infusion set type. Using an incorrect infusion rate could result in using an infusion rate that is too high or too low.

For example, if the user is using an infusion set that has a single needle, the user must make sure to use the proper infusion rate associated with single site infusion rather than infusion into two sites. When infusing into a single site, incorrectly using the rate associated with two sites instead of one can result in infusion of the medication at a rate that is too high for a single site, which could lead to patient discomfort. In another example, if the user is using an infusion set that has two needles, the user must make sure to use the proper infusion rate associated with two infusion sites rather than just one. When infusing into two sites, incorrectly using the rate associated with a single site instead of two sites can result in longer infusion times and/or incomplete delivery of the full medication dosage.

Some embodiments provide an infusion pump for administering medication to a patient that automatically determines whether an infusion set that is coupled to the infusion pump is configured to infuse medication at one site or at two sites on the patient's body. The infusion pump may use the determined configuration (e.g., one site or two sites) of the infusion set to determine a duration of time for completing infusion of the medication into the patient, and then control the pump engine to infuse the medication into the patient over the determined duration of time. For example, when the infusion pump is set to infuse the medication at one site, the total rate at which the medication can be infused into the patient may be less than when the infusion pump is set to infuse the medication at two sites. The infusion pump may use a determination of the configuration of the infusion set to control the pump engine (e.g., determine and/or control rates of infusion).

An infusion pump for infusing medication into a patient may undergo a priming stage in order to prepare the infusion set to infuse the medication into a patient. An amount of the medication may be pumped from a source container and through an infusion set via which the infusion pump infuses medication into the patient. Conventional infusion pumps require a user to manually prime the infusion set with an amount of medication prior to infusion. For example, a user may draw an amount of medication into the infusion set by manually pulling back on a syringe. The inventors have recognized that priming often requires movement of the medication to precise locations in the infusion set, and thus manual priming may be time consuming and/or difficult for those with limited dexterity. For example, priming may require that an amount of medication be moved through the infusion set up to an infusion needle without actually entering the needle.

Some embodiments provide an infusion pump for administering medication to a patient that aids a user in priming an infusion set. The infusion pump can activate the pump engine to cause an amount of medication to advance from a source container through the infusion set at one or more flow rates. The rate(s) may allow a user to move the medication to specific points in the infusion set that may be difficult to do manually.

When infusing medication into a patient using an infusion pump, it may be necessary to check a placement of one or more needles in the body of the patient. For example, in subcutaneous therapy, it may be necessary to confirm that the needle(s) have not been inserted into one or more blood vessels.

The inventors have recognized that having an infusion pump configured to aid in checking for proper needle placement can aid in facilitating infusion. Some embodiments provide an infusion pump for administering medication to a patient that automatically pulls back fluid so the user can check for proper placement of one or more needles in a body of the patient. The infusion pump may be configured to control a pump engine that is configured to infuse medication into the patient. The infusion pump may operate in (1) a delivery mode in which fluid is drawn from a source container through an inlet of the infusion set towards an outlet of the infusion set, and (2) a reverse mode in which the pump engine draws fluid from the outlet towards the inlet. The infusion pump may operate in the reverse mode to draw fluid from a patient so the user can determine whether blood was drawn from the patient by visually inspecting the infusion set tubing near the needle. The infusion pump eliminates the need for a patient to manually draw back fluid to confirm proper placement of the needle(s), which the inventors have recognized to be time-consuming and difficult for patients with limited dexterity.

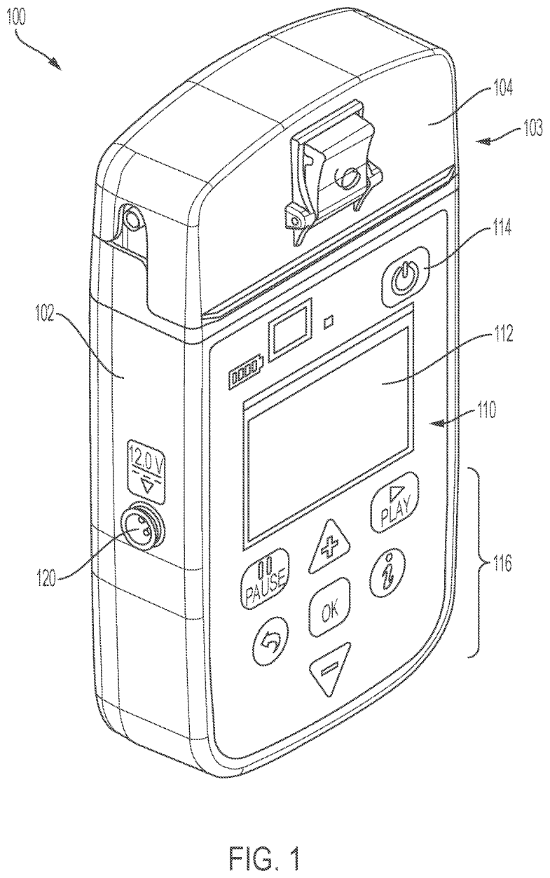

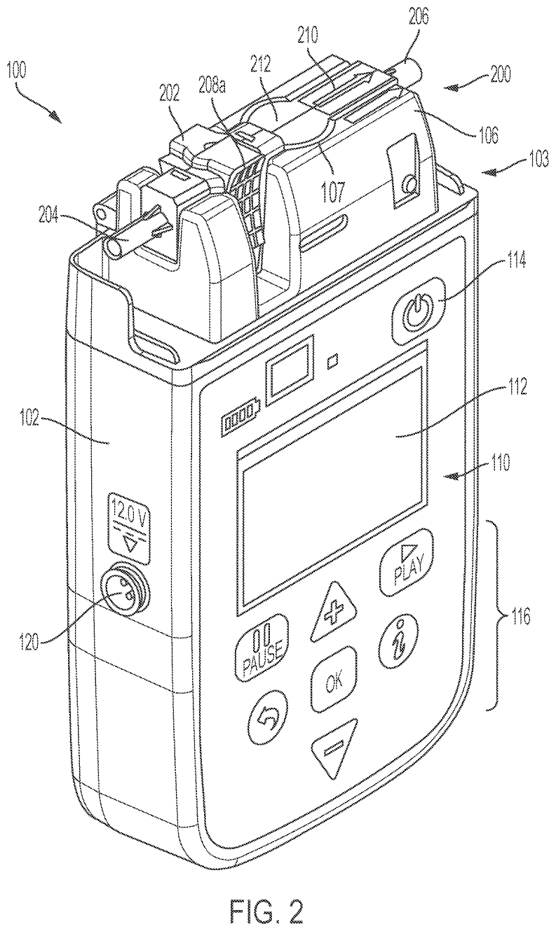

FIG. 1 shows a perspective view of an embodiment of an infusion pump 100 having a housing 102 and a user interface 110 as well as a pump engine (for example, see pump engine 200 in FIG. 2). The infusion pump 100 may be configured to infuse medication into a patient. The housing 102 includes a compartment 103 and contains internal components such as a motor and controller. The compartment 103 is covered by an openable compartment lid 104 which is configured to receive a pump engine when opened and secure a pump engine when closed. The user interface 110 includes a display 112, a power switch 114 configured as a power button, and user controls 116. The display 112 is configured to convey information to a user of the infusion pump 100, such as pumping status, instructions for use, alerts, warnings, or any other desirable information. The power button 114 may be used to supply power to a controller and/or motor. As shown in FIG. 1, the infusion pump 100 includes a power connector 120 for connecting the infusion pump 100 to a power source configured to supply a source of electrical power. In some embodiments, the infusion pump 100 may include an internal battery which may be used to selectively supply power to the controller and motor. In some embodiments, the user controls 116 may be used to program or select different operational modes of the infusion pump 100, respond to prompts, or otherwise interact with the controller.

In some embodiments, the infusion pump 100 may include a computer system. For example, the computer system may include one or more processors, memory, and storage.

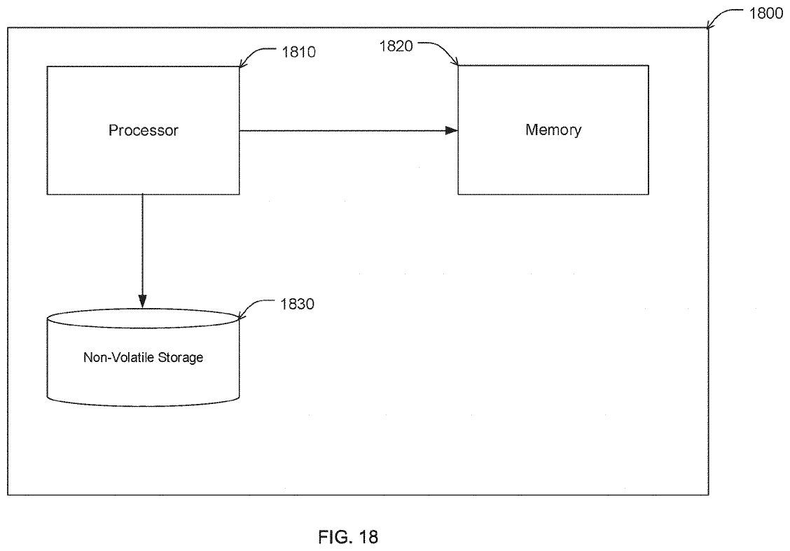

An example computer system which may form part of the infusion pump 100 is described below with reference to FIG. 18.

FIG. 2 depicts a perspective view of the infusion pump 100 of FIG. 1 with the compartment lid 104 removed. As shown in FIG. 2, the compartment 103 includes a receptacle 106 configured to receive and secure a pump engine 200. The pump engine 200 includes a housing 202, an inlet 204, and an outlet 206. In some embodiments, the pump engine 200 includes a rotor disposed in the housing 202 configured to rotate and move fluid from the inlet 204 to the outlet 206. The rotor may be configured as any suitable positive displacement rotor or centrifugal pump rotor. As shown in FIG. 2, the pump engine 200 is non-symmetrical and includes a finger grip 208a and an alignment portion 212 which are configured to interact with the receptacle 106 to align the pump engine 200 for operative connection between the pump engine 200 and the infusion pump 100. That is, the receptacle 106 is shaped to receive and orient the pump engine 200 in a correct orientation for alignment between a pump coupling and a motor coupling. The receptacle 106 is shaped to complement the shape of the pump engine 200 with the finger grip 208a and the alignment portion 212. Accordingly, the receptacle 106 may prevent the reception of the pump engine 200 unless the pump engine 200 is in a correct orientation, thereby promoting consistent and reliable operative connection between the pump engine 200 and the infusion pump 100.

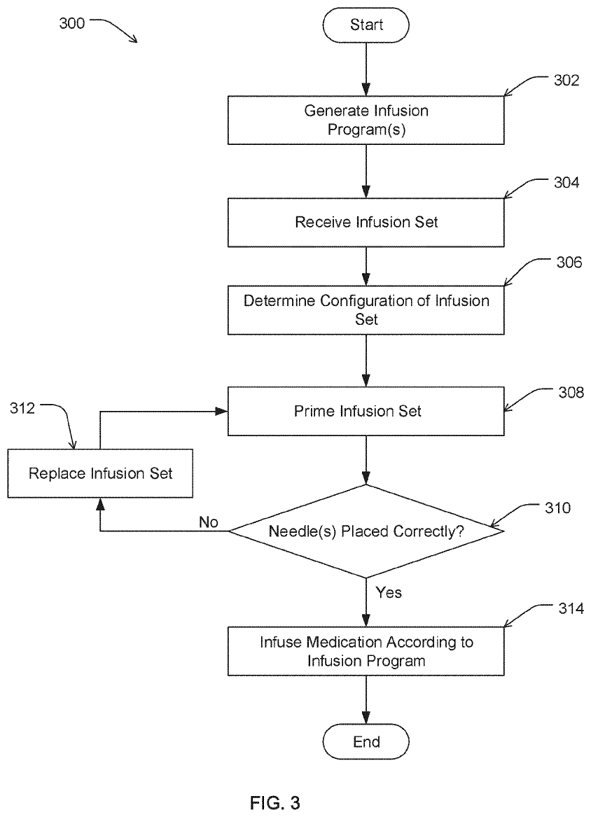

FIG. 3 shows a process 300 for infusing medication into a patient according to an aspect of the present application. Process 300 may be performed by any computing device(s) comprising one or more processors. For example, process 300 may be performed by infusion pump 100 described above with reference to FIG. 1.

Process 300 begins at block 302 where the infusion pump performing process 300 generates time intervals and rates at which to infuse one or more medications into a patient. The generated time intervals and rates may be referred to herein as an “infusion program.” In some embodiments, the duration for each time interval may be user-specified. In some embodiments, the infusion program may calculate and assign infusion rates for each time interval. The calculation may be made based on the requirements of the medication to be delivered and/or the condition to be treated. For example, the infusion program may include a first interval of 5 minutes, a second interval of 10 minutes, and a third interval of 10 minutes. The infusion program may assign an infusion rate of 5 mL/hour to the first interval, a rate of 10 mL/hour to the second interval, and a rate of 20 mL/hour to the third interval. Some embodiments are not limited to any number of intervals, length of intervals, or rates described herein, as values provided herein are for illustrative purposes. The infusion pump may execute the infusion program to perform infusion of the medication(s) for the specified time intervals at the rate associated with each time interval. In some embodiments, the infusion program may be a sequence of time intervals with associated rates at which to infuse the medication(s) into a patient. The infusion pump may use the infusion program to perform infusion of the medication(s) according to the sequence.

In some embodiments, the infusion pump may be configured to receive one or more user inputs specifying information based on which the infusion pump generates the infusion program(s). The infusion pump may use the user input(s) to generate an infusion program that is customized for a particular patient, a particular medication, and/or a particular type of infusion. For example, the infusion pump may receive an indication of weight of the patient, a total dosage of the medication(s) that the patient is to receive, how many times the patient has received the infusion in the past, and/or a type of medication that the infusion pump is to infuse. The infusion pump may generate the infusion program(s) based on the received inputs.

In some embodiments, the infusion pump may be configured to generate the infusion program(s) without receiving user inputs. In some embodiments, the infusion pump may be configured to generate the infusion program(s) based on information stored in a memory of the infusion pump. For example, the infusion pump may store information about the patient, infusion history of the patient, and/or one or more medications that are to be infused, and generate the infusion program(s) based on the stored information.

After generating the infusion program(s) at block 302, process 300 proceeds to block 304 where the infusion pump receives an infusion set. The infusion set may be plugged into a receptacle (e.g., receptacle 106 as seen in FIG. 2) of the infusion pump. The infusion set may include a pump engine (e.g., pump engine 200, as seen in FIG. 2) that is controlled by the infusion pump. The infusion pump may be configured to control the pump engine to infuse medication into a patient via the infusion set. In some embodiments, the infusion set includes an administration set and a needle set. In some embodiments, the administration set includes tubing and the pump engine. In some embodiments, the needle set includes tubing and one or more needle assemblies that each includes a needle that may be inserted into a site on a body of the patient. The infusion pump may be configured to control the pump engine to pump medication from a source container through the administration set and through the needle set to infuse medication into the patient via the needle(s).

Process 300 proceeds to block 306 where the infusion pump determines whether the infusion set is configured to infuse medication via one site on the patient's body, or multiple sites (e.g., two) on the patient's body. In some embodiments, different types of infusion sets may be configured to administer medication at a different number of sites. For example, the infusion pump may be configured to receive a first type of infusion set that is configured to infuse medication at one site. The first type of infusion set may have a single needle assembly with a single needle that delivers medication to a patient at a single site on the body of the patient. The infusion pump may further be configured to receive a second type of infusion set that is configured to infuse medication at two sites. The second type of infusion set may include two needle assemblies that each have a needle. The two needles may be inserted into the patient at two different sites. The infusion pump may be configured to control the pump engine to infuse medication into the patient via both sites simultaneously. In some embodiments, the infusion pump may be configured to receive infusion sets that are configured to infuse medication at three, four, five, six, and/or seven sites, and may have three, four, five, six, and/or seven needles.

Process 300 proceeds to block 308 where the infusion pump primes the infusion set for infusing medication into the patient. In some embodiments, the infusion pump may be configured to draw one or more medications from respective source containers into one or more portions of an infusion set received by the infusion pump at block 304. For example, the infusion pump may control the pump engine to pump the medication(s) through the infusion set up to one or more needle assemblies without entering the needle(s).

Process 300 proceeds to block 310 where the infusion pump determines whether one or more needles have been placed correctly at sites on the body of the patient. For example, the infusion pump may determine whether the needle(s) were inserted into blood vessel(s) of the patient. In this scenario, it may be necessary to replace the needle(s) and re-insert them into the site(s). The infusion pump may be configured to perform an automated needle check procedure to determine if the needle(s) have been inserted into a blood vessel. In some embodiments, a pump engine controlled by the infusion pump may be operable in a reverse mode in which the infusion pump can draw fluid out from a patient via the needle(s). The infusion pump may be configured to operate the pump engine in reverse mode to cause the pump engine to draw fluid from the patient.

In some embodiments, the infusion pump may aid the user in determining whether the needle(s) were placed correctly based on whether blood is drawn into the needle. In some embodiments, the infusion pump may be configured to determine whether blood is drawn into the needle using a sensor (e.g., an optical sensor). In some embodiments, the infusion pump may be configured to determine whether blood is drawn into the needle based on user input. If the infusion pump determines that blood is drawn in to the needle, the infusion pump may determine that the needle(s) are not placed correctly and process 300 proceeds to block 312 where the infusion set is replaced. For example, the infusion pump may instruct the user to replace the infusion set with a new infusion set and re-insert the needle(s). If the infusion pump determines that no blood has entered the infusion set, the infusion pump may determine that the needle(s) have been placed correctly and process 300 proceeds to block 314 where the infusion pump infuses medication according to the infusion program generated at block 302. In some embodiments, the infusion pump may be configured to automatically infuse medication into the patient by controlling a pump engine based on the time intervals and rates. For example, the infusion pump may be configured to initiate and modulate a motor that operates a pump engine to draw medication from a respective source container and pump the medication through the infusion set into the patient at one or more sites. After infusing the medication at block 314, process 300 ends.

In some embodiments, an infusion pump is not limited to performing each and every one of the processes described above with reference to FIG. 3. In some embodiments, the infusion pump may be configured to perform one or more of the steps and not the other steps. For example, the infusion pump may be configured to automatically determine the type of infusion set coupled to the infusion pump as described with reference to block 306, but may not be configured to aid in priming a pump engine as described with reference to block 308. Some embodiments are not limited to any particular combination of functions described above with reference to FIG. 3.

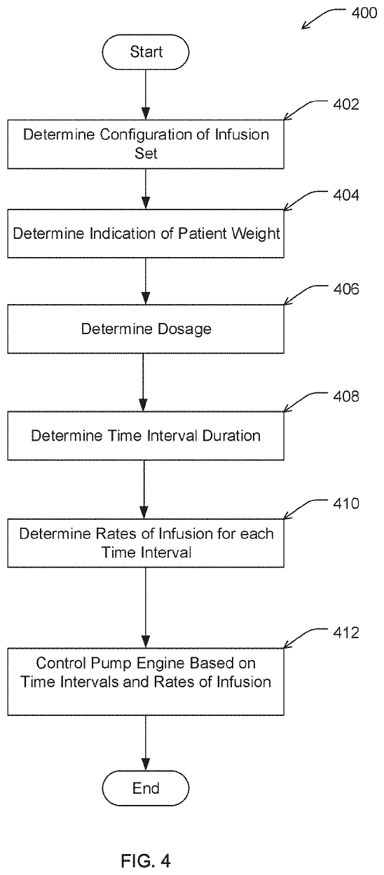

FIG. 4 shows a process 400 for generating an infusion program according to an aspect of the present application. Process 400 may be performed by any computing device(s) comprising one or more processors. For example, process 400 may be performed by infusion pump 100 described above with reference to FIG. 1.

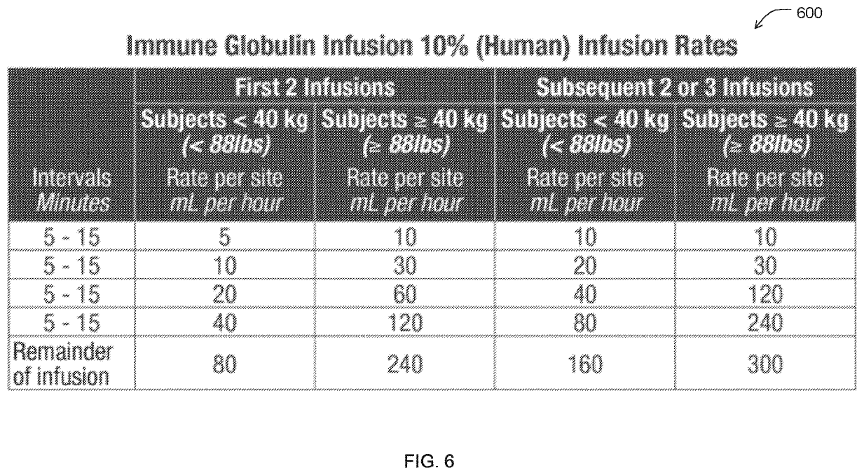

Process 400 begins at block 402 where the infusion pump performing process 400 determines the type of infusion set that has been inserted into the infusion pump. In some embodiments, the infusion pump may be configured to determine whether the infusion set is configured to deliver medication via one site, or via multiple sites (e.g., two). For example, a user may insert an infusion set into the receptacle 106 of infusion pump 100 as shown in FIG. 2. A first type of infusion set may be configured to deliver medication at one site (e.g. by having a single needle), while a second type of infusion set may be configured to deliver medication at two sites (e.g. by having two needles). An example technique for determining the configuration of the infusion set is described below with reference to FIG. 6.

Process 400 proceeds to block 404 where the infusion pump determines an indication of patient weight. The infusion pump may determine the indication of patient weight by prompting a user for the information. In some embodiments, the infusion pump may provide the user with various weight ranges and ask a user to select an appropriate weight range for the patient. In some embodiments, the infusion pump may prompt a user to input the specific weight of the patient.

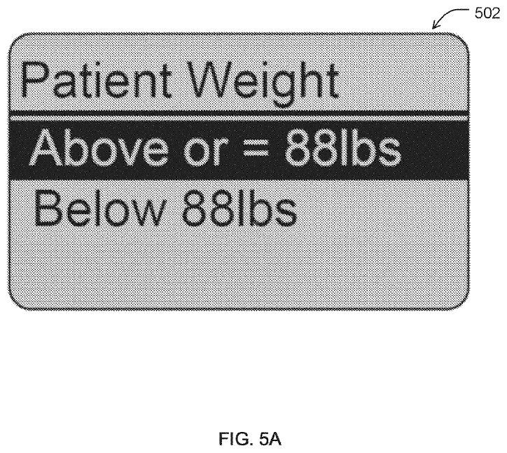

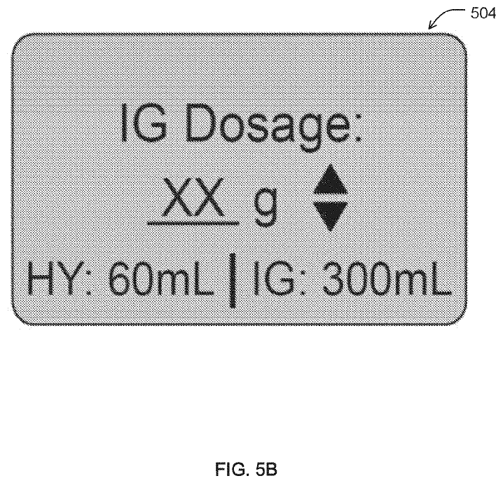

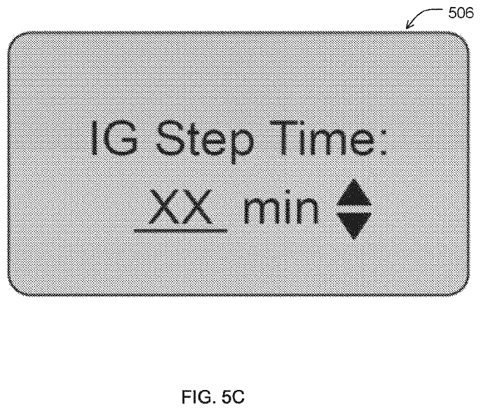

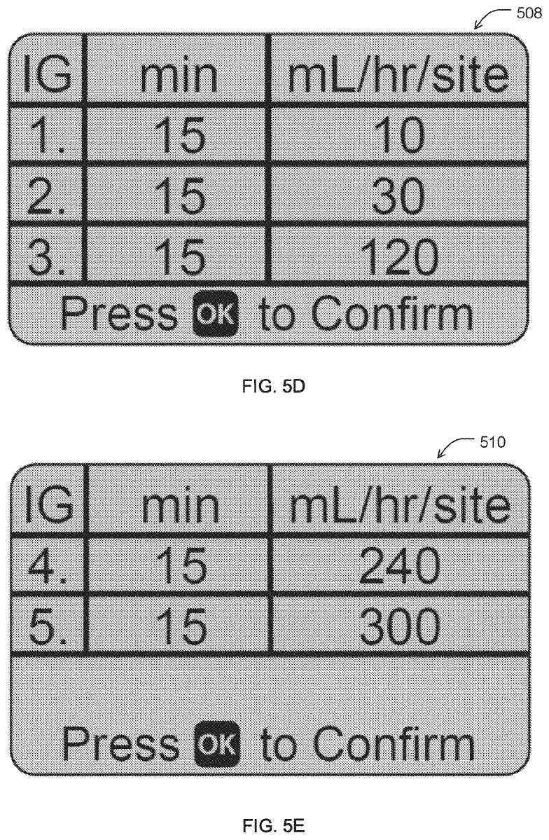

In some embodiments, the infusion pump may be configured to generate, on a display of the infusion pump, a user interface screen that prompts a user to enter the indication of the patient weight. FIG. 5A shows an example user interface screen 502 that allows the user to select a range that includes the patient weight. For example as shown in user interface screen 502, the user may make a first selection indicating that the weight of the patient is above a threshold weight (e.g., 88 lbs.) or a second selection indicating that the weight of the patient is below the threshold weight. In some embodiments, the infusion pump may generate a user interface screen on the display that prompts the user to enter a specific weight value.

In some embodiments, the infusion pump may be configured to determine the indication of weight without a user input. In some embodiments, the infusion pump may be configured to access a stored indication of weight for the patient. For example, the infusion pump may determine the indication of the weight based on a stored weight of the patient in a memory of the infusion pump. The stored weight may have been entered by a caregiver, and stored for subsequent infusions. In some embodiments, the infusion pump does not require the user to re-enter patient weight during subsequent infusions.

Process 400 proceeds to block 406 where the infusion pump determines a dosage of medication that is to be infused into the patient. In some embodiments, the infusion pump may be configured to determine a total amount of medication that is to be infused into the patient. For example, the infusion pump may be configured to determine a total mass of the medication that is to be infused into the patient. In another example, the infusion pump may be configured to determine a total volume of medication that is to be infused into the patient. In some embodiments, the infusion pump may be configured to generate, on a display of the infusion pump, a user interface screen via which the infusion pump may receive a user input specifying a total amount of the medication that is to be infused into the patient. For example, the infusion pump may receive a value of a total mass and/or volume entered by the user. FIG. 5B shows an example user interface screen 504 via which the infusion pump may receive a user input specifying a dosage of the medication. In screen 504, the user can specify a total mass of the medication that is to be infused into the patient.

In some embodiments, the infusion pump may be configured to automatically determine a dosage without receiving user input. In some embodiments, the infusion pump may be configured to access a data store to determine the dosage that the patient is to be infused with. For example, a caregiver may have entered a dosage into the infusion pump which is then stored for subsequent infusions of the medication.

Process 400 proceeds to block 408 where the infusion pump determines a time interval length of infusion. In some embodiments, the infusion pump determines the time interval length by receiving an input from a user specifying the time interval length. The time interval length is a duration of time for which the infusion pump is to infuse medication into the patient at a particular rate. For example, the infusion pump may be configured to determine a number of seconds or minutes for which the infusion pump is to maintain one or more rates of infusion of medication. In some embodiments, the infusion pump may be configured to generate, on a display of the infusion pump, a user interface screen via which the infusion pump receives a user input specifying the time interval duration. FIG. 5C illustrates an example user interface screen 506 via which the infusion pump may receive a user input specifying the time interval duration. The user interface screen 506 allows a user to enter a specific value of the duration.

In some embodiments, the infusion pump may be configured to determine a time interval length without receiving a user input. In some embodiments, the infusion pump may be configured to access a time interval length value stored in a memory of the infusion pump. In some embodiments, the infusion pump may be configured to access a value entered for the patient in a previous infusion of the medication. The infusion pump may then use the previously entered value. In some embodiments, the time interval length is pre-set and cannot be changed by the user.

Process 400 proceeds to block 410 where the infusion pump calculates infusion rates for each time interval. In some embodiments, the infusion pump may be configured to determine a rate at which medication is to be infused into the patient for each of the time intervals. In some embodiments, the infusion pump may be configured to use the determined information at blocks 402-408 to calculate appropriate infusion rates. In some embodiments, the infusion pump may be configured to store data specifying time interval lengths for different values of the information determined by the infusion pump. FIG. 6 shows a table 600 illustrating example data that may be stored by the infusion pump, and accessed to calculate appropriate infusion rates. For example, if at blocks 402-408 the infusion pump determined that the patient is greater than 88 lbs., and that the time interval duration should be 15 minutes, the infusion pump sets an infusion sequence comprising a series of five 15 minute infusion intervals, with respective rates of 10, 30, 120, 240, and 300 mL/hr/site. In some embodiments, the infusion pump may be configured to generate, on a display of the infusion pump, one or more user interface screens displaying the generated intervals and rates such as screens 508-510 displayed FIGS. 5D-E. For example, the infusion pump may generate a table displaying the time intervals and associated infusion rates.

In some embodiments, the infusion pump may be configured to store data (e.g., data illustrated in table 600) used for determining the timer intervals and lengths in an internal storage of the infusion pump. For example, the infusion pump may store the data in memory.

In some embodiments, the infusion pump may be configured to determine the infusion rates in units of mL per hour per site (mL/hr/site). By determining an infusion rate per site, the infusion pump may control a pump engine to infuse medication using infusion sets of different types that have different numbers of infusion sites. For example, if the infusion pump determines that the infusion set is of the first type (e.g., having a single needle) at block 402, the infusion pump may control the pump engine to infuse medication at a single site at the specified rates of mL/hr/site. In another example, if the infusion pump determines that the infusion set is of the second type (e.g., having two needles) at 402, the infusion pump may control the pump engine to infuse medication at two sites at the specified rates of mL/hr/site. When infusing into two sites, the infusion pump may control the pump engine to infuse medication at a total infusion rate that is twice the rate of the specified mL/hr/site rate in order to deliver the correct rate to each site. For example, in order to infuse at a rate of 10 mL/hr/site using two infusion sites, the infusion pump may control the pump engine to output at a rate of 20 mL/hr.

After determining the time intervals and rates at block 410, process 400 proceeds to block 412 where the infusion pump controls a pump engine (e.g., pump engine 200 as seen in FIG. 2) based on the determined time intervals and rates of infusion. In some embodiments, the infusion pump may be configured to modulate a throttle of a motor to control the rate at which the pump engine is moving the medication through the infusion set. In some embodiments, the infusion pump may be configured to use a timer based on which the infusion pump modifies infusion rates according to the intervals and rates determined at block 410. The infusion pump may initiate the timer when the infusion pump initiates infusion of the medication. For example, the infusion pump may initiate infusion at a rate of 10 mL/hr/site for the infusion program shown in FIGS. 5D-E. In response to determining, based on the timer, that 15 minutes have passed, the infusion pump may increase the infusion rate from 10 mL/hr/site to 30 mL/hr/site. Subsequently, when the infusion pump determines that another 15 minutes have passed based on the timer, the infusion pump may control the pump engine to increase the infusion rate from 30 mL/hr/site to 120 mL/hr/site. In some embodiments, the infusion pump may continue infusion based on a determined infusion program until a dosage determined at block 406 has been infused into the patient. The infusion pump may calculate a total amount (e.g., volume and/or mass) that has been infused into the patient. The infusion pump may compare the total amount to the dosage specified at block 406. The infusion pump may stop infusion if it detects that the amount infused meets the dosage determined at block 406.

In some embodiments, the infusion pump may be configured to change a rate at which the medication is being infused into the patient to a different value from one specified by a determined infusion program. In some embodiments, the infusion pump may be configured to receive a user input specifying a change in the rate at which medication is being infused. For example, the patient may experience an intolerable amount of pain when infusing at a particular rate of infusion, and wish to increase or decrease the rate. In some embodiments, the infusion pump may be configured to receive user input commanding a change in rate of infusion. For example, the infusion pump may include one or more buttons that, when pushed, command a change in rate of infusion. In some embodiments, the infusion pump may be configured to modify the current rate of infusion in response to receiving a user input commanding a change in the rate of infusion. For example, a user may press a button commanding a decrease in the rate of infusion and, in response to detecting that the button has been pressed, the infusion pump may decrease a current rate of infusion.

In some embodiments, the infusion pump may be configured to modify one or more subsequent time intervals and/or associated rates of infusion as a result of changing a current rate of infusion. For example, if the infusion pump decreased a rate of infusion at a first time interval, the infusion pump may extend a length of one or more subsequent time intervals to ensure that a total dosage of the medication is infused into the patient. In some embodiments, the infusion pump may be configured to prevent modification of a rate of infusion beyond a threshold rate. For example, the infusion pump may be configured to prevent increasing a current rate of infusion beyond a maximum rate. In another example, the infusion pump may be configured to prevent decreasing a current rate of infusion below a minimum rate. In some embodiments, the infusion pump may be configured to allow only decreasing of a current rate of infusion. For example, the infusion pump may be configured to reject a user's command to increase a current rate of infusion. In some embodiments, the infusion pump may have a setting in which a user is prohibited from modifying the current rate of infusion. For example, any command to modify the current rate of infusion would be rejected by the infusion pump.

After the infusion pump controls the infusion pump to infuse medication into the patient at block 412, process 400 ends.

In some embodiments, as part of process 400, the infusion pump will determine how many times the patient has received the infusion in the past. The infusion pump may determine this information by, for example, prompting the user to input the information, or by accessing stored infusion history in memory. The infusion pump may use the infusion history information in its process of determining proper infusion rates.

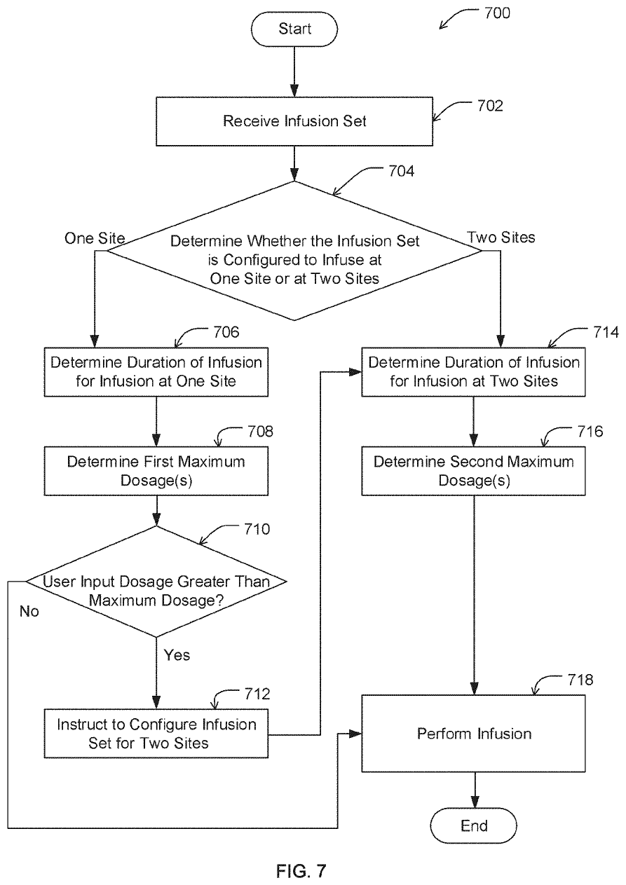

FIG. 7 shows a process 700 for configuring an infusion pump according to a determined configuration of an infusion set according to an aspect of the present application. Process 700 may be performed by any computing device(s) comprising one or more processors. For example, process 700 may be performed by infusion pump 100 described above with reference to FIG. 1.

Process 700 begins at block 702 where the infusion pump receives an infusion set. For example, a patient may insert an infusion set into the receptacle 106 of infusion pump 100 as shown in FIG. 2. In some embodiments, the infusion set may be of a first type that is configured to deliver medication at one site, or of a second type of infusion set that is configured to deliver medication at two sites. The first type of infusion set may have a single needle assembly with a single needle. The patient may insert the single needle at one site on the body of the patient. When the infusion pump controls a pump engine (e.g., pump engine 200 as seen in FIG. 2) to infuse the medication using an infusion set of the first type, the infusion pump may control the pump engine to pump medication into the patient via the needle at the single site. The second type of infusion set may have two needle assemblies, each with a needle. The patient may insert the two needles at two different sites on the body of the patient. When the infusion pump controls the pump engine to infuse the medication using an infusion set of the second type, the infusion pump may control the pump engine to pump medication into the patient via both of the two needles at the two sites.

Process 700 proceeds to block 704 where the infusion pump determines whether the infusion set received by the infusion pump is configured to infuse medication at one site or at two sites. In some embodiments, the infusion pump may be configured to determine whether the infusion pump should be configured to infuse medication at one site or at two sites by determining a type of infusion set received by the infusion pump. If the infusion pump determines that the received infusion set is of the first type, the infusion pump may determine that the infusion set is configured to deliver the medication at one site. If the infusion pump determines that the received infusion set is of the second type, the infusion pump may determine that the infusion set is configured to deliver the medication at two sites.

In some embodiments, an infusion set includes a pump engine that includes a housing, an inlet, and an outlet. The inlet and the outlet may be pre-connected to fluidic connectors and/or a needle set. The infusion set may have a particular type depending on the characteristics of an administration set and needle set that form the infusion set. For example, a needle set may be singular, bifurcated, trifurcated, and/or have particular needle sizes that change the fluid flow properties (e.g., flow rate, turbulence, viscous resistance, etc.) of the infusion set. In another example, an administration set may include fluidic connectors which determine (1) which types of infusion pumps that the infusion set can be coupled to, and (2) the fluid flow characteristics of the infusion set. Accordingly, it may be desirable to determine the type of infusion set automatically to activate or modify one or more operational modes of an infusion pump.

In some embodiments, the infusion pump may be configured to determine the type of infusion set based on a pump engine of the infusion set. A particular pump engine which is coupled to an infusion set may include at least one infusion set indicator disposed in or on a housing of the pump engine in a predetermined position. In some embodiments, the infusion pump may include one or more sensors disposed in predetermined locations in the infusion pump that are configured to sense one or more infusion set indicators in proximity to the sensor(s). The infusion pump may be configured to determine the type of the infusion set based on how many infusion set indicator(s) are sensed and/or the relative positioning of the infusion set indicator(s).

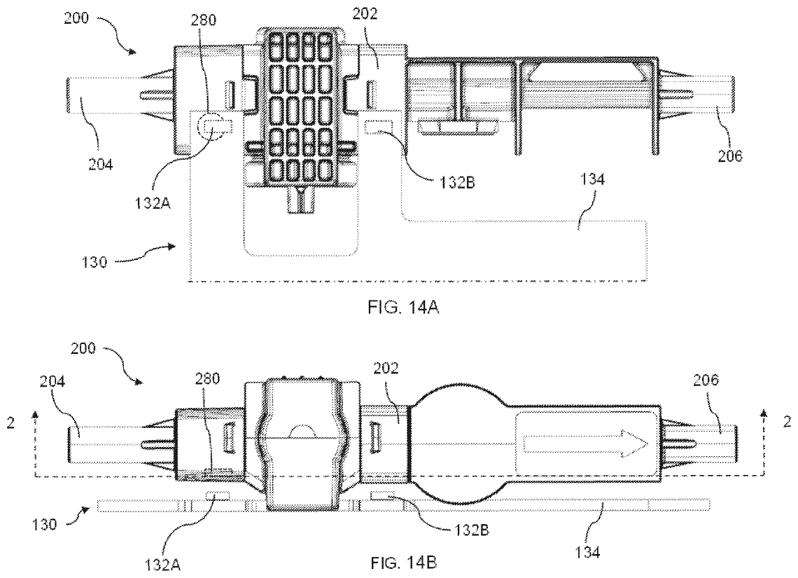

FIGS. 14A-14B show front and top views of an embodiment of a pump engine 200 and infusion set sensor assembly 130 according to an aspect of the present application. As shown in FIG. 14A, the pump engine 200 includes a housing 202, an inlet 204, and an outlet 206. The pump engine 200 includes a single infusion set indicator 280 disposed within the housing 202. The infusion set indicator is detectable by the infusion set sensor assembly 130 which may be disposed in the infusion pump. The infusion set sensor assembly includes first and second infusion set sensors 132A, 132B which are configured to detect the presence of one or more infusion set indicators disposed in or on the housing the pump engine. The infusion set sensors 132A-B are disposed on a PCB 134 which may supply power and data communications to each of the infusion set sensors. In some embodiments, the infusion set indicator 280 is configured as a magnet and the infusion set sensors 132A, 132B are Hall Effect sensors which detect the presence of the magnetic field of the magnetic infusion set indicator. The infusion set indicator may be any suitable detectable portion of the pump engine, including, but not limited to, optical indicators, radioactive indicators, and/or magnetic indicators.

In some embodiments, the sensors 132A-B may be used by the infusion pump to detect the type of infusion set by determining the presence of one or more infusion indicators on the pump engine 200. For example, the first infusion set sensor 132A detects the infusion set indicator 280 when the pump engine 200 is brought into proximity and alignment with the infusion set sensor assembly as illustrated in FIG. 14B. The infusion pump may determine that the infusion set is of a particular type based on input from the sensor 132A indicating that the infusion set indicator 280 was detected, and/or input from the sensor 132B indicating that an indicator has not been detected. The infusion set indicator 280 may be positioned on the pump engine 200, different than the one shown in FIG. 14B, such that when the pump engine 200 is received by the infusion pump, the infusion set indicator 280 is brought into proximity and alignment with the second infusion set sensor 132B. The infusion pump may determine that the infusion set is of a different type when the infusion pump receives an indication from the second infusion set sensor 132B indicating that the infusion set indicator 280 was detected than when the infusion pump receives an indication from the first infusion set sensor 132A indicating that the infusion set indicator 280 was detected. In some embodiments, one type of infusion set may have indicators present at multiple locations on the pump engine 200, while a different type of infusion set may have an indicator present at a single location on the pump engine 200. The infusion pump may be configured to determine the type of infusion set based on whether input received from the sensors 132A-B indicates that presence of an infusion set indicator has been detected by both sensors 132A-B, or that presence of an infusion set indicator has been detected by one of the sensors 132A-B.

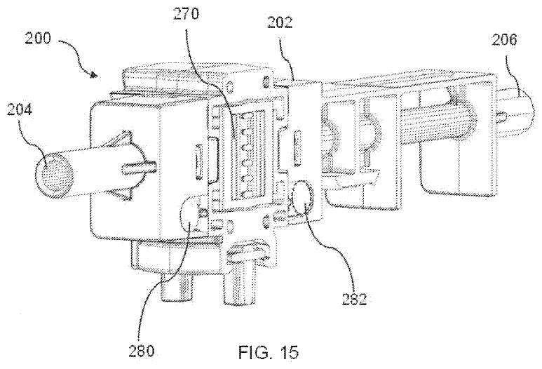

FIG. 15 shows a perspective cross-sectional view of the pump engine 200 of FIGS. 14A-B taken along line 2-2 of FIG. 26B. As shown in FIG. 15 and discussed previously, the pump engine includes a housing 202, an inlet 204, and an outlet 206. The pump engine 200 includes a rotor 270 disposed in the housing which is arranged to move fluid from the inlet 204 to the outlet 206 or from the outlet 206 to the inlet 204 when the rotor is rotated. Also disposed in the housing is an infusion set indicator 280 which is configured as a magnet. In some embodiments, the infusion set indicator 280 is integrated into the housing 202 on an inlet-side of the rotor. The housing 202 also has space for an optional second infusion set indicator 282 on the outlet-side of the rotor. As discussed herein, each infusion set indicator may be detectable by at least one infusion set sensor of an infusion pump. In some embodiments, the at least one infusion set sensor is configured as a Hall Effect sensor which is arranged to detect the magnetic field of the infusion set indicator 280 and/or infusion set indicator 282 when the sensor is in proximity to the infusion set indicator.

In some embodiments, the position and number of the infusion set indicator(s) 280, 282 detected by the sensor(s) may be used by the infusion pump to determine a type of infusion set. For example, if the infusion set indicator 280 is positioned on the inlet-side of the rotor 270 as shown in FIG. 15, the pump engine may be part of an infusion set configured to medication at two sites (e.g., having a bifurcated needle set). In this example, if the second infusion set indicator 282 is positioned on the outlet-side of the rotor without the infusion set indicator 280 on the inlet-side of the rotor, the pump engine 200 may be part of an infusion set configured to infuse medication at one site (e.g., having a singular needle set). In some embodiments the pump engine 200 may include both the first infusion set indicator 280 and the second infusion set indicator 282, where the presence of both indicators is associated with another distinct type of infusion set (e.g., trifurcated needle set). In some embodiments, the pump engine 200 may be configured with any suitable number of infusion set indicators in any suitable position such that the type of an infusion set may be determined by the infusion pump.

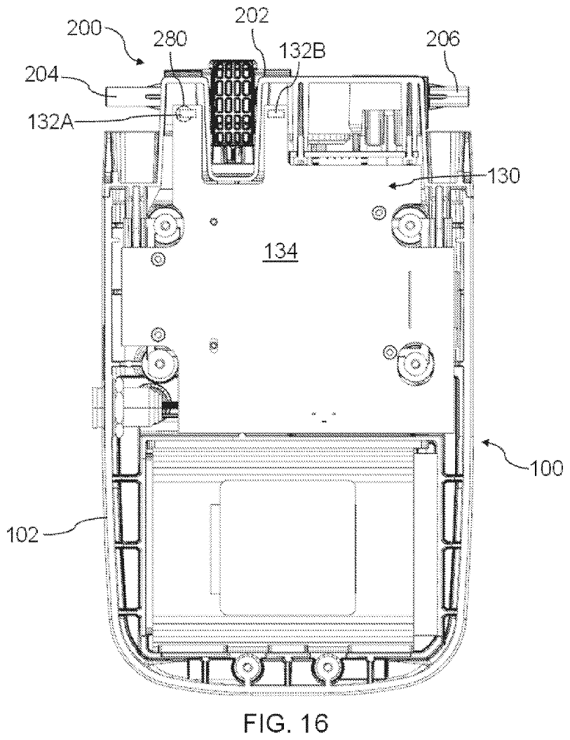

FIG. 16 shows an embodiment of a pump engine 200 including an infusion set indicator 280 disposed in an embodiment of a control unit 100 of an infusion pump 100 including an infusion set sensor assembly 130. As shown in FIG. 16, the pump engine 200 includes a housing 202, an inlet 204, and an outlet 206. The infusion set indicator 280 is disposed on an inlet-side of a rotor (e.g., as shown in FIG. 27) and is fully enclosed in the housing. The infusion set sensor assembly 130 is positioned inside of a housing 102 of the infusion pump 100 and includes a first infusion set sensor 132A and a second infusion set sensor 132B disposed on a PCB 134 between the PCB 134 and the pump engine 200. While in FIG. 15 the infusion set sensors are shown disposed on a PCB 134, the infusion set sensors may be mounted independent of any PCB or other elements, as the present disclosure is not so limited. The first and second infusion set sensors 132A-B are configured to detect the presence of the infusion set indicator 280 when the infusion set indicator 280 is in proximity with the infusion set sensors 132A-B. The infusion set sensors 132A-B are configured as any suitable sensor for detecting the presence of the infusion set indicator. For example, the infusion set indicator 280 may be magnetic and the infusion set sensors 132A-B may be Hall Effect sensors. As another example, the infusion set indicator 280 may be optically distinct from the rest of the pump unit and detectable by infusion set sensors 132A-B configured as optical sensors (e.g., laser scanner, camera-based reader, color sensor, etc.).

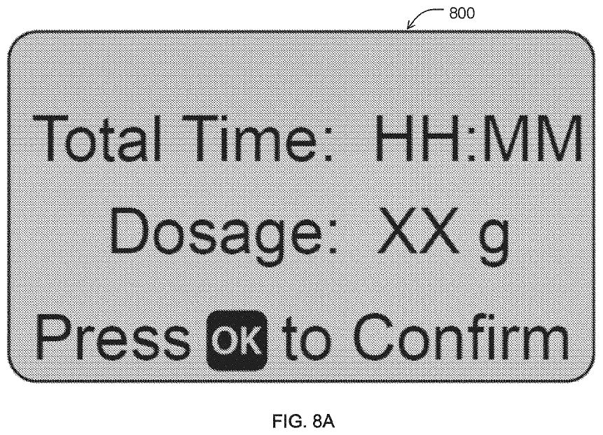

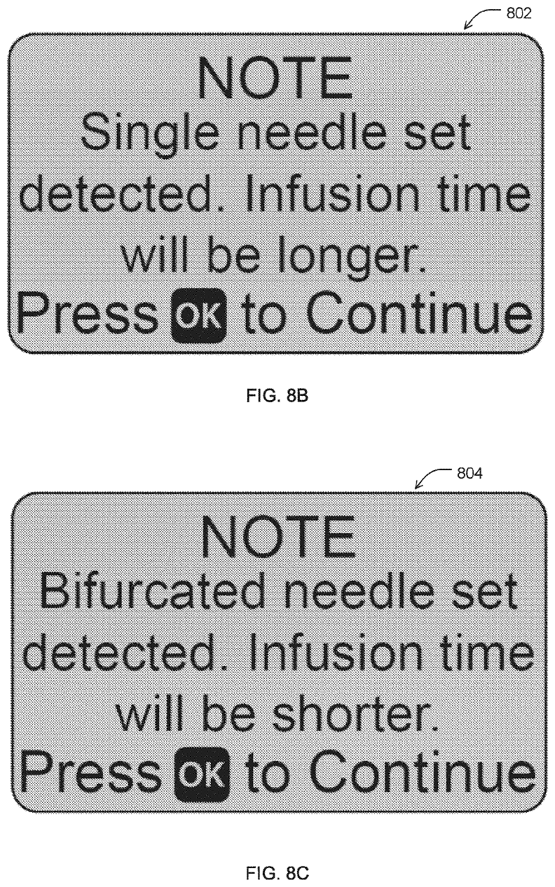

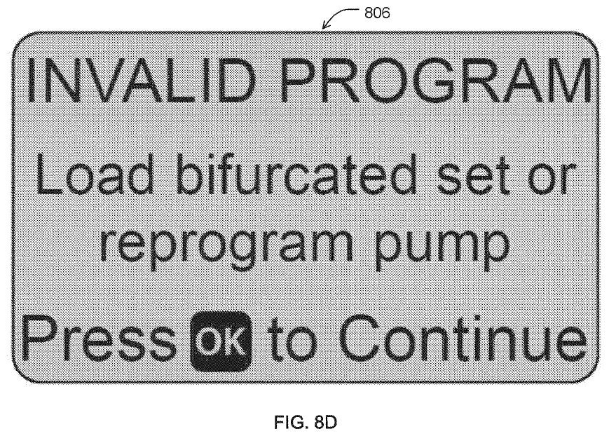

In some embodiments, the infusion pump may be configured to receive a user input specifying a type of infusion set that is to be used for infusion of the medication. For example, the infusion pump may generate, on a display (e.g., display 112 as seen in FIG. 1) of the infusion pump, a user interface screen via which the infusion pump may receive a user selection of a type of infusion set to be used for infusion of the medication. In some embodiments, the infusion pump may be configured to compare the type of infusion set specified by the user input to a type of infusion set detected using the sensor. In some embodiments, the infusion pump may be configured to generate an indication of a discrepancy if the infusion pump determines that the type of infusion set detected using the sensor is different from the type of infusion set specified by the user input. For example, the infusion pump may be configured to generate, on the display of the infusion pump, a user interface screen notifying the user of the detected discrepancy. FIG. 8B shows an example user interface screen 802 that may be generated by the infusion pump if the infusion pump detects that the infusion set is of the first type but the user input specifies an infusion set of the second type. The screen 802 indicates that infusion time may be longer as the medication will only be infused into the patient via one site. FIG. 8C shows an example user interface screen 804 that may be generated by the infusion pump if the infusion pump detects that the infusion set is of the second type but the user input specifies an infusion set of the first type. The screen 804 indicates that infusion time may be shorter as the medication will be infused into the patient simultaneously at two sites. Thus the overall rate of infusion may be greater than that of one site.

In some embodiments, the infusion pump may receive both a user input of infusion set type, while also determining on its own which type of infusion set has been coupled to the infusion pump. In this manner, the two methods of determining the infusion type may serve as a check on one another. In some embodiments, if the two methods arrive at a different result, the infusion pump may prompt a user to visually double check whether the coupled infusion set has a single needle or two needles, and to re-input the infusion set type into the pump.

In some embodiments, the infusion pump may be configured to prevent infusion of the medication if the infusion pump determines that the detected type of infusion set is different from the type specified by the user input. For example, a doctor or other medical care provider may have provided the user input based on an intended method of treatment. The infusion pump, by comparing a detected type of infusion set to the user input, may ensure that a patient using the infusion pump is adhering to the treatment prescribed by the doctor or other medical care provider.

If at block 704, the infusion pump determines that the infusion set is configured to infusion the medication at one site, process 700 proceeds to block 706 where the infusion pump determines a length of time for infusing the medication at one site. In some embodiments, the infusion pump may be configured to determine a length of time for infusing medication at one site based on an infusion program. In some embodiments, the infusion pump may calculate a total time of infusion based on one or more time intervals and one or more associated rates determined by the process during process 400 described above with reference to FIG. 4. For example, the infusion pump may calculate a total length of time that the infusion pump will control the pump engine to infuse medication into the patient based on the determined total dosage, time interval durations, and associated rates of infusion per site.

Process 700 proceeds to block 708 where the infusion pump determines a maximum dosage of the medication that the patient may receive when the infusion set is configured to infuse the medication at one site. The patient may be able to receive a maximum amount of the medication through a site over a time period. The infusion pump may determine the maximum dosage of the medication as the maximum amount of the medication that the patient can receive through a single site over a time period.

In some embodiments, the infusion pump may be configured to generate an indication of a determined length of time for infusion and/or the determined maximum dosage. In some embodiments, the infusion pump may be configured to generate, on a display of the infusion pump (e.g., display 112 as seen in FIG. 1), a user interface screen shown the determined length of time and the maximum dosage. FIG. 8A shows an example user interface screen 800 that may be generated by the infusion pump displaying a length of time and maximum dosage. The user interface screen may request user input to confirm that the user is aware of the determined length of time and the determined maximum dosage.

Process 700 proceeds to block 710 where the infusion pump determines whether a user-specified dosage of medication received by the infusion pump is greater than the maximum dosage determined at block 708. For example, the infusion pump may determine whether a dosage value received by the infusion pump via a user interface screen generated by the infusion pump is possible to infuse into the patient through one site. In some embodiments, the infusion pump may be configured to compare the dosage specified by a user input to the maximum dosage determined at block 708.

If at block 710 the infusion pump determines that the received dosage is greater than the maximum dosage when the infusion set is configured to infuse the medication at one site, process 700 proceeds to block 712 where the infusion pump instructs the user to configure the infusion set for two sites. In some embodiments, the infusion pump may be configured to generate, on the display of the infusion pump, an indication of an instruction to change the infusion set in response to determining that the user specified dosage is greater than the maximum dosage. FIG. 8D shows an example user interface screen 806 which the infusion pump may generate on the display of the infusion pump in the case the that the infusion pump detects the discrepancy. User interface screen 806 indicates that the infusion set does not comply with the infusion program (e.g., a maximum dosage), and displays instructions to load an infusion set of a different type. When the infusion pump receives an infusion set that is configured to infuse medication at two sites (e.g., an infusion set of the second type), process 700 proceeds to block 714 where the infusion pump determines the duration of infusion for an infusion set configured to infuse medication at two sites as described below.

If at block 710 the infusion pump determines that the received dosage is less than the maximum dosage when the infusion set is configured to infuse medication at one site, process 700 proceeds to block 718 where the infusion pump performs infusion of the medication. In some embodiments, the infusion pump may continue to a process of priming the infusion set for infusion of the medication, performing a check to determine whether the needle has been placed correctly at the site, and/or controlling the pump engine to infuse the medication into the patient.

Returning again to block 704, if the infusion pump determines that the infusion set is configured to infuse medication at two sites, process 700 proceeds to block 714 where the system determines a duration of time for infusing medication at two sites. In some embodiments, the infusion pump may be configured to determine a duration of time for infusing medication at two sites based on an infusion program. For example, the infusion pump may calculate a total time of infusion based on one or more time intervals and one or more associated rates determined by the process during process 400 described above with reference to FIG. 4. The infusion pump may calculate a total length of time that the infusion pump will control the pump engine to infuse medication into the patient based on the determined total dosage, time intervals, and associated rates of infusion per site specified by the infusion program. In some embodiments, the determined length of time for infusing the medication at two sites may be less than the length of time for infusing the medication at one site determined at block 706. The infusion pump may control the pump engine to infuse medication to two sites at rates specified by the infusion program. As a result, the overall rate of infusion at two sites may be greater than that of infusion at one site. In some embodiments, the duration of time for infusion at two sites may be approximately half of the duration of time for infusion at one site.

Process 700 proceeds to block 716 where the infusion pump determines a maximum dosage of medication that can be received by the patient when the infusion set is configured to infuse medication at two sites. In some embodiments, the infusion pump may be configured to determine a maximum dosage that can be infused at two sites that is greater than the maximum dosage that can be infused at one site determined at block 708. In some embodiments, the maximum dosage for an infusion set configured to infuse at two sites may be approximately twice as much as the maximum dosage for an infusion set configured to infuse at one site.

In some embodiments, the infusion pump may be configured to verify that a user-specified dosage of medication that is to be infused is less than the maximum allowed dosage determined at block 716. In some embodiments, the infusion pump may be configured to prevent infusion of the medication if the user specified dosage is greater than the maximum allowed dosage determined at block 716. The infusion pump may generate, on a display of the infusion pump, one or more user interface screens indicating that the entered dosage is greater than the maximum dosage. The infusion pump may request that the user enter a different dosage. In some embodiments, the infusion pump may be configured to ensure that a dosage of medication that is infused into the patient meets a limit for safety of the patient and/or to meet prescription requirements of the medication.

In some embodiments, the infusion pump may be configured to generate an indication of a determined length of time for infusion and/or the determined maximum dosage. In some embodiments, the infusion pump may be configured to generate, on the display of the infusion pump, a user interface screen shown the determined length of time and the maximum dosage. FIG. 8A shows an example user interface screen 800 that may be generated by the infusion pump displaying a length of time and maximum dosage. The user interface screen may request user input to confirm that the user is aware of the determined time duration and maximum dosage.

Process 700 proceeds to block 718 where the infusion pump performs infusion of the medication through two sites. In some embodiments, the infusion pump may be configured to prime the infusion set for infusion, check placement of the two needles at the two sites, and/or control the pump engine to infuse the medication into the patient at the two sites.

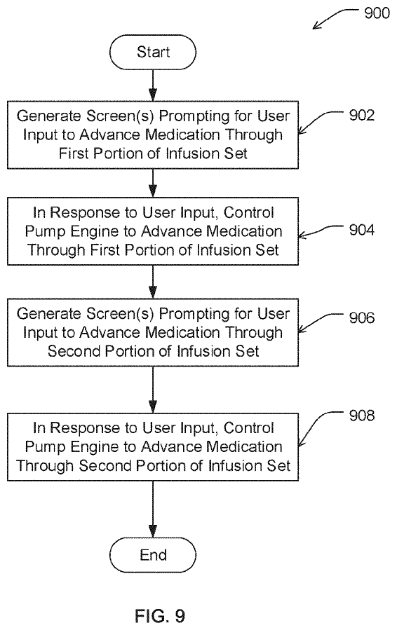

FIG. 9 shows a process 900 for priming an infusion set for infusing medication into a patient according to an aspect of the present application. Process 900 may be performed by any computing device(s) comprising one or more processors. For example, process 90 may be performed by infusion pump 100 described above with reference to FIG. 1.

Process 900 may be performed by the infusion pump to prepare an infusion set to infuse medication into a patient. In some embodiments, process 900 may be performed after the infusion pump receives an infusion set via which the infusion pump is to infuse the medication into the patient. For example, the infusion set may be inserted into a receptacle 106 of the infusion pump 100 as shown in FIG. 2.

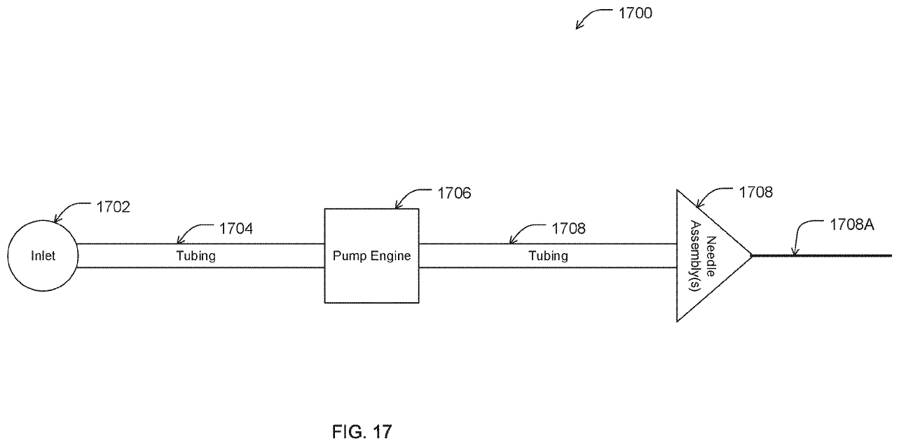

FIG. 17 shows a schematic diagram of an infusion set 1700 according to an aspect of the present application. The infusion set 1700 includes an inlet 1702, tubing 1704 between the inlet 1702 and a pump engine 1706, tubing 1708 between the pump engine 1706 and one or more needle assemblies 1708. Each needle assembly 1708 may include a needle 1708A which may be inserted into the patient. In some embodiments, an infusion pump may be configured to operate the pump engine 1706 to pump medication from a source container through the infusion set. The medication enters the infusion set 1700 via the inlet 1702 and travels through the tubing 1704 towards the pump engine 1706. The medication is then pumped through tubing 1708 and into each of the one or more needle assemblies 1708. In some embodiments, a first portion of the infusion set 1700 may comprise the inlet 1702, the tubing 1704, and the pump engine 1706. In some embodiments, a second portion of the infusion set 1700 may comprise the tubing 1708, and the one or more needle assemblies 1708.

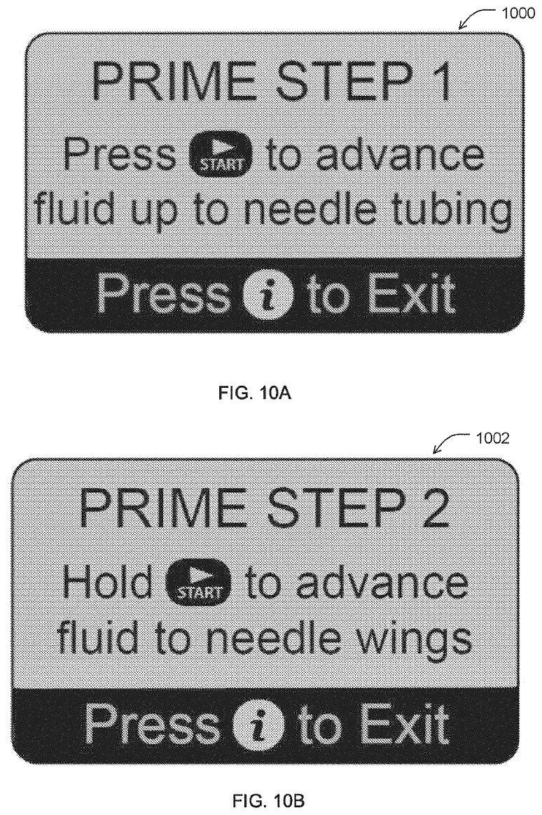

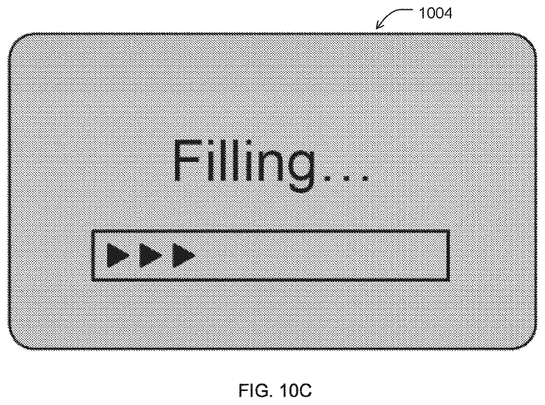

Process 900 begins at block 902 where the infusion pump generates, on a display of the infusion pump (e.g., display 112 as seen in FIG. 1), one or more user interface screens prompting for user input to advance medication through a first portion of the received infusion set. In some embodiments, the first portion of the infusion set may be the administration set of the infusion set. For example, the first portion may be from an inlet of the infusion set to the outlet 206 of a pump engine 200 of the infusion set. In some embodiments, the infusion pump may undergo a first step of priming in which the infusion pump is to advance an amount of medication through the first portion of the infusion set. FIG. 10A shows an example of a user interface screen 1000 instructing the user to provide a user input to advance the medication through a first portion of the infusion set in a first step.

Process 900 proceeds to block 904 where the infusion pump receives a user input and, in response to the user input, controls the pump engine to advance an amount of the medication through the first portion of the infusion set. For example, the infusion pump may be configured to control the pump engine by controlling a throttle of a motor that powers the pump engine to advance the amount of medication from a source container through the first portion of the infusion set. In some embodiments, the infusion pump may be configured to control the pump engine to advance the medication through the administration set without entering a subsequent portion of the infusion set.

In some embodiments, the infusion pump may be configured to control the pump engine to advance medication through the first portion of the infusion set in response to detecting that a button on the infusion pump was pressed. In some embodiments, the button may be a physical button on the infusion pump which, when pressed, triggers generation of a signal that indicates to the infusion pump that the button has been pressed. For example, the press of the button may trigger a change in a voltage signal based on which the infusion pump detects that the button has been pressed. In some embodiments, the infusion pump may control the pump engine to advance medication through the first portion of the infusion set in response to detecting an input received via a touch screen display of the infusion pump. For example, the infusion pump may advance the medication in response to detecting a tap, or press and hold of an option displayed on the touch screen display.

In some embodiments, the infusion pump may be configured to continuously advance the medication from the source container through the first portion while receiving the user input. For example, the infusion pump may continue to advance the medication while a button is pressed, or while an option on a touch screen is held. The infusion pump may stop advancing the medication from the source container through the first portion when the infusion pump no longer receives the user input. For example, the infusion pump may stop advancing the medication if the infusion pump detects that a button is no longer pressed, or an option in a user interface displayed on a touch screen is no longer selected.

In some embodiments, the infusion pump may be configured to trigger advancing of the medication from the source container through the first portion in response to the user input. The infusion pump may be configured to automatically stop advancing medication through the first portion of the infusion set after the amount of medication has reached an end of the first portion. For example, the infusion pump may determine that the amount of medication has reached a juncture between the administration set and the needle set of the infusion set and, in response, stop advancing the medication. In some embodiments, the infusion pump may be configured to automatically stop advancing the medication by determining a quantity (e.g., a volume) of the medication that has been drawn from the container. When the determined quantity reaches a threshold value, the infusion pump may control the pump engine to stop advancing the medication. The quantity of medication may be an amount that fills the first portion of the infusion set.

In some embodiments, the infusion pump may be configured to advance the medication through the first portion of the infusion set at a first rate. In some embodiments, the infusion pump may be configured to control the pump engine to advance the medication through the first portion of the infusion set at approximately 500-1500, 700-1200, 800-1000, or 850-950 mL/hr.

After advancing the medication through the first portion of the infusion set, process 900 proceeds to block 906 where the infusion pump generates one or more user interface screens prompting a user for input to advance the medication through a second portion of the infusion set. In some embodiments, the second portion of the infusion set may be the needle set. For example, the second portion of the infusion set may be from an inlet of the needle set up to one or more needles of the needle set. In some embodiments, the infusion pump may undergo a second step of priming in which the infusion pump advances an amount of medication through the second portion of the infusion set. FIG. 10B shows an example of a user interface screen 1002 instructing the user to provide a user input to advance the medication through the second portion of the infusion set in the second step.

Process 900 proceeds to block 908 where the infusion pump receives a user input and, in response to the user input, controls the pump engine to advance an amount of the medication through the second portion of the infusion set. For example, the infusion pump may be configured to control the pump engine by controlling a throttle of a motor that powers the pump engine to advance the amount of medication through the second portion of the infusion set. In some embodiments, the infusion pump may be configured to control the pump engine to advance the medication through the needle set without entering needles of one or more needle assemblies of the infusion set.

In some embodiments, the infusion pump may be configured to control the pump engine to advance medication through the second portion of the infusion set in response to detecting that a button on the infusion pump was pressed. In some embodiments, the button may be a physical button on the infusion pump which, when pressed, triggers generation of a signal that indicates to the infusion pump that the button has been pressed. For example, the press of the button may trigger a change in a voltage signal based on which the infusion pump detects that the button has been pressed. In some embodiments, the infusion pump may be configured to control the pump engine to advance medication through the first portion of the infusion set in response to detecting an input on a touch screen display of the infusion pump. For example, the infusion pump may advance the medication in response to detecting a tap, or press and hold of an option displayed on the touch screen display.

In some embodiments, the infusion pump may be configured to continuously advance the medication from the source container through the second portion while receiving the user input. For example, the infusion pump may continue to advance the medication while a button is pressed, or while an option on a touch screen is pressed. The infusion pump may stop advancing the medication through the second portion when the infusion pump no longer receives the user input. For example, the infusion pump may stop advancing the medication if the infusion pump detects that a button is no longer pressed, or an option in a user interface displayed on a touch screen is no longer pressed.

In some embodiments, the infusion pump may be configured to trigger advancing of the medication from the source container through the second portion in response to the user input. The infusion pump may be configured to automatically stop advancing medication through the second portion of the infusion set after the amount of medication has reached an end of the second portion. For example, the infusion pump may determine that the amount of medication has reached one or more needle assemblies. In some embodiments, the infusion pump may be configured to automatically stop advancing the medication by determining a quantity (e.g., a volume) of the medication that has been drawn from the container. When the determined quantity reaches a threshold value, the infusion pump may control the pump engine to stop advancing the medication. The quantity of medication may be an amount that fills the second portion of the infusion set as required for a second step of priming. For example, the amount of medication may be an amount that fills the second portion of the infusion set with medication without advancing the medication into one or more needle assemblies.

In some embodiments, the infusion pump may be configured to advance the medication through the second portion of the infusion set at a second rate different from the first rate at which the medication was advanced through the first portion of the infusion set. In some embodiments, the infusion pump may be configured to control the pump engine to advance the medication through the second portion of the infusion set at a rate of approximately 100-500, or 200-300 mL/hr. In some embodiments, the second rate at which medication is advanced through the second portion of the infusion set is slower than the first rate at which medication is advanced through the first portion of the infusion set. The slower rate of the second step may allow a user to more easily visualize an amount of the medication advancing through the second portion, and to stop the medication from advancing after reaching a particular point. For example, the user may be able to stop the medication from advancing into one or more needles.

In some embodiments, the infusion pump may be configured to generate a screen on a display of the infusion pump (e.g., display 112 as seen in FIG. 1) indicating that the infusion pump is advancing the medication through the infusion set. FIG. 10C shows an example user interface screen 1004 of a status display indicating that the infusion pump is controlling the pump engine to advance medication through the infusion set. In the, user interface screen 1004 displays a “Filling . . . ” message that indicates of advancing the medication. In some embodiments, the infusion pump may be configured to generate the user interface screen in response to receiving one or more user inputs that initiate advancing of medication. For example, the infusion pump may be configured to generate the user interface screen in response to detecting a button is pressed. In some embodiments, the infusion pump may be configured to generate the user interface screen in response to operation of the pump engine to pump the medication from a source container. For example, the infusion pump may generate the user interface screen when the throttle speed of a motor used to power the infusion pump reaches a threshold value.

After the medication has been advanced through the second portion of the infusion at block 908, the priming process 900 ends. For example, the infusion pump may proceed to control the pump engine to infuse the medication into the patient after performing the priming process 900.

Although process 900 describes a priming procedure that is performed in two steps, some embodiments may include more than two priming steps. In some embodiments, one of the first or second portion of priming described above may be separated into multiple portions. For example, the first step may be divided into two steps. In some embodiments, the infusion pump may be configured to advance medication through a portion (e.g., an administration set and/or needle set) of the infusion set in multiple steps. The infusion pump may be configured to advance medication through a different section of the portion of the infusion set during each of the steps. In some embodiments, the infusion pump may be configured to modify the rate at which the medication is advanced through different sections of the portion of the infusion set.

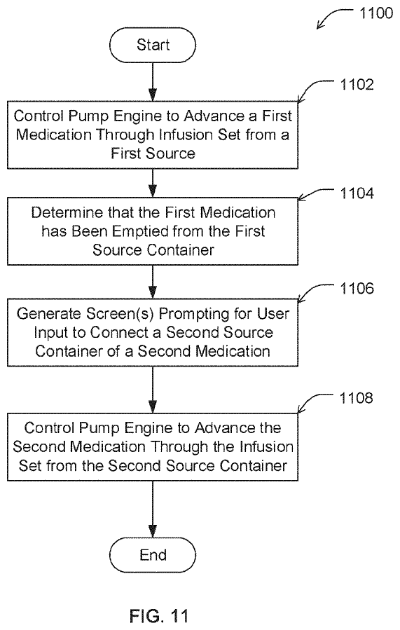

FIG. 11 shows a process 1100 for priming an infusion set for infusing two types of medication into a patient according to an aspect of the present application. Process 1100 may be performed by any computing device(s) comprising one or more processors. For example, process 1100 may be performed by infusion pump 100 described above with reference to FIG. 1.

Process 1100 begins at block 1102 where the infusion pump controls a pump engine (e.g., pump engine 200 as seen in FIG. 2) to advance an amount of a first medication through the infusion set from a first source container. In some embodiments, the infusion pump may be configured to control the pump engine to advance the first medication through the infusion set in response to receiving a user input. For example, the infusion pump may detect pressing of a button of the infusion pump, or selection of a selectable option displayed on a touch screen display. In response, the infusion pump may control the pump engine to pump the first medication from the first container through the infusion set. In some embodiments, the infusion pump may be configured to pump the first medication through the infusion set as described above with reference to FIG. 9.

Process 1100 proceeds to block 1104 where the infusion pump determines that the first medication has emptied from the first source container. In some embodiments, the infusion pump may be configured to determine that the first medication has emptied from the source container by determining that there is no more fluid flowing through the infusion set. For example, the infusion pump may include a flow meter that indicates a flow rate of fluid flow through the infusion set. The infusion pump may determine that the first medication has emptied from the source container when the flow rate is approximately zero. In some embodiments, the infusion pump may be configured to use an optical detector and/or a pressure sensor may detect that liquid is no longer flowing through tubing. In some embodiments, the infusion pump may be configured to determine that the first medication has emptied from the first container based on a user input. For example, the infusion pump may receive a user selection indicating that the first container has emptied.

Process 1100 proceeds to block 1106 where the infusion pump generates one or more screens instructing a user to connect a second source container that holds a second medication. In some embodiments, the infusion pump may be configured to generate a user interface screen on a display of the infusion pump instructing the user to connect a second source container holding the second medication. For example, the infusion pump may be configured to display the user interface screen(s) on the display 100.

Process 1100 proceeds to block 1108 where the infusion pump controls the pump engine to advance the second medication through the infusion set. The infusion pump controls the pump engine to pump an amount of the second medication from the second source container through the infusion set. In some embodiments, the infusion pump may be configured to control the pump engine to advance the second medication through the infusion set in response to receiving a user input. For example, the infusion pump may detect a button of the infusion pump pressed, or a selectable option displayed on a touch screen display of the infusion pump selected. In response, the infusion pump may control the pump engine to pump the second medication from the second source container through the infusion set. In some embodiments, the infusion pump may be configured to pump the second medication through the infusion set as described above with reference to FIG. 9.

After advancing the amount of the second medication through the infusion set, process 2100 ends. For example, the infusion pump may proceed to one or more subsequent steps such as checking needle placement, and/or controlling the pump engine to infuse the two medications into the patient.

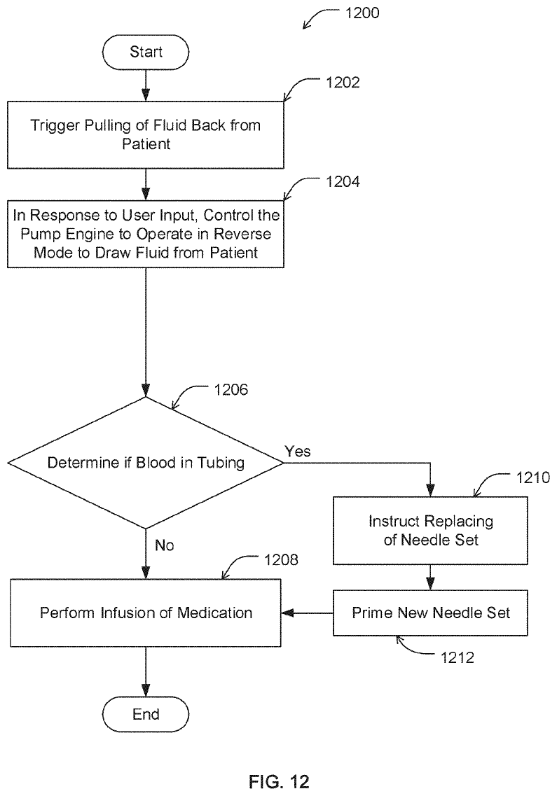

FIG. 12 shows a process 1200 for determining whether one or more needles have been placed correctly on a patient's body according to an aspect of the present application. Process 1200 may be performed by any computing device(s) comprising one or more processors. For example, process 1200 may be performed by infusion pump 100 described above with reference to FIG. 1.

Process 1200 may be performed after one or more needles have been placed at one or more sites on a patient. For example, a user may have inserted the needle(s) into the site(s) on the patient's body. Process 1200 may be performed to aid the user in ensuring that the needle(s) were inserted properly at the site(s). For example, process 1200 may be performed to check whether the needle(s) were mistakenly inserted into one or more blood vessels in a vicinity of the site(s).

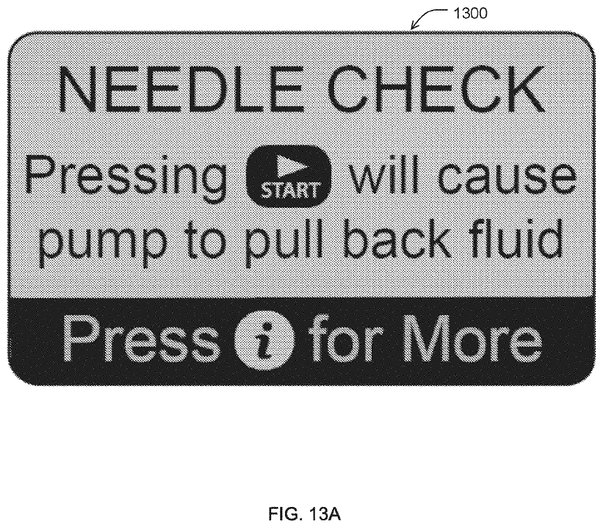

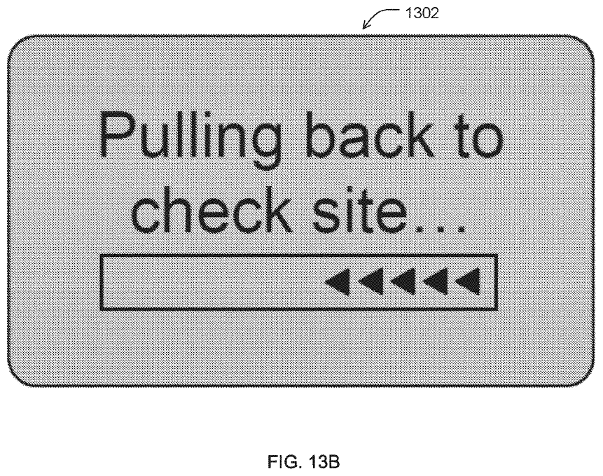

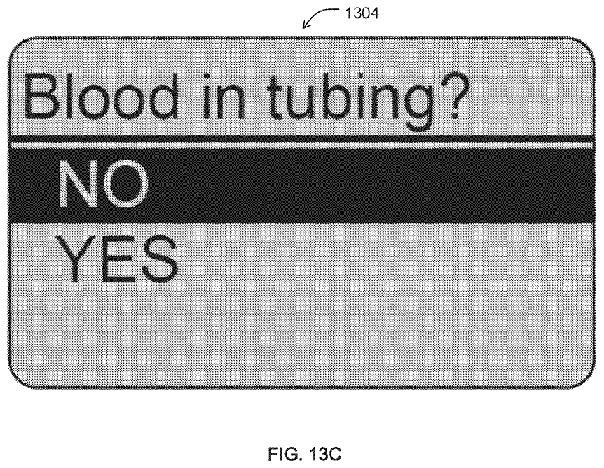

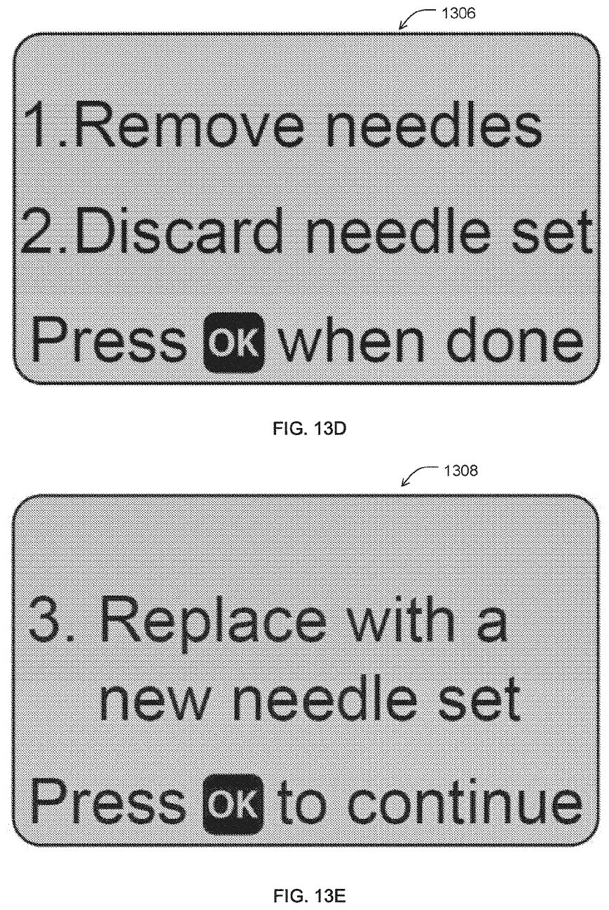

Process 1200 begins at block 1202 where the infusion pump triggers pulling of fluid back from the patient. In some embodiments, the infusion pump may be configured to generate one or more user interface screens prompting for user input to initiate pulling of fluid back from a patient, through the needle set tubing. In some embodiments, the infusion pump may be configured to display the user interface screen(s) on a display of the infusion pump. FIG. 13A shows an example user interface screen 1300 that may be generated and displayed by the infusion pump. User interface screen 1300 includes a message instructing a user of the infusion pump to press a start button or option to cause the infusion pump to control a pump engine (e.g., pump engine 200 as seen in FIG. 2) to pull fluid back from the patient.

Process 1200 proceeds to block 1204 where the infusion pump receives a user input and, in response, controls the pump engine to operate in reverse mode to draw fluid from the patient. In some embodiments, the infusion pump may operate in multiple modes. The multiple modes may include a first mode (also referred to as a “delivery mode”) in which the infusion pump pumps medication from a source container, through an infusion set, and into a patient. The multiple modes may include a second mode (also referred to as a “reverse mode”) in which the infusion pump pumps fluid from a patient (e.g., via the needle(s) inserted into the patient) and through the infusion set. In response to the user input, the infusion pump may operate the pump in the reverse mode. For example, to control the mode of operation of the pump engine, the infusion pump may be configured to control a direction of operation of motor 180 as seen in FIGS. 7A-B.

In some embodiments, the infusion pump may be configured to generate, on a display of the infusion pump (e.g., display 112 as seen in FIG. 1), a user interface screen indicating that the pump engine is being operated in the reverse mode. FIG. 13B shows an example user interface screen 1302 that may be generated by the infusion pump indicating of operation in the reverse mode. User interface screen 1302 shows a message indicating that the infusion pump is operating the pump engine to pull fluid back from the patient to perform a site check.

Process 1200 proceeds to block 1206 where the infusion pump determines whether there is blood in tubing of the infusion set after operating the pump engine in the reverse mode. In some embodiments, the infusion pump may be configured to determine whether there is blood in the tubing by receiving user input indicating whether there is blood in the tubing. For example, the infusion pump may generate, on a display of the infusion pump, one or more user interface screens requesting user input whether there is blood in the tubing of the infusion set. FIG. 13C shows an example user interface screen 1304 requesting user input that indicates whether there is blood in the tubing. The user interface screen 1304 asks a user if there is blood in the tubing and provides “no” selectable option and a “yes” selectable option. The infusion pump may determine whether there is blood in the tubing of the infusion set based on the response.

In some embodiments, the infusion pump may be configured to determine whether there is blood in the tubing of the infusion set automatically. The infusion pump may include a sensor that senses fluid flowing from the patient and into the infusion set. For example, the sensor(s) may be flow rate sensor that detects a change in the flow rate (e.g., a reverse) that indicates fluid flow in the reverse direction. The infusion pump may use the sensor to determine if there is blood in the tubing of the infusion set. For example, if the infusion pump determines that there was flow of fluid in the reverse direction, the infusion pump may determine that there is blood in the tubing.

If at block 1206 the infusion pump determines that there is no blood in the tubing, process 1200 proceeds to block 1208 where the infusion pump performs infusion of the medication into the patient. In some embodiments, the infusion pump may be configured to control the pump engine to pump medication from a source container through an infusion set to infuse the medication into the patient.

If at block 1206 the infusion pump determines that there is blood in the tubing, process 1200 proceeds to block 1210 where the infusion pump instructs replacement of a needle set (or, in some embodiments, the entire infusion set). For example, one or more needles of the needle set may have been inserted into blood vessel(s) of the patient. In some embodiments, the infusion pump may be configured to generate, on the display of the infusion pump, one or more user interface screens instructing a user to replace the needle set. FIGS. 13D-E show user interface screens 1306-1308 instructing a user to remove one or more needles, discarding the needle set(s) or the entire infusion set, and replacing the needle set with a new needle set (or replacing the entire infusion set with a new infusion set).

In some embodiments, the infusion pump may be configured to determine when the needle set/infusion set has been replaced. In some embodiments, the infusion pump may determine when the needle set has been replaced based on receiving a user input. For example, the infusion pump may detect a pressing of a button, or selection of an option in a touch screen display based on which the infusion pump determines that the needle set has been replaced. In one example, user interface screen 1308 requests a user input of pressing “OK.” In response to the user input, the infusion pump may determine that the needle has been replaced.

After the needle set has been replaced at block 1210, process 1200 proceeds to block 1212 where the infusion pump primes the new needle set. In some embodiments, the infusion pump may be configured to prime the new needle set as described in steps 1206-1208 discussed above with reference to FIG. 9. For example, the infusion pump may be configured to control the pump engine to pump an amount of medication that is to be infused into the patient through the needle set. In some embodiments, after the needle set has been replaced at block 1210, the infusion pump may be configured to repeat a process of checking the needle. For example, the infusion pump may be configured to return to block 1202 of process 1200.

After priming the new needle set at block 1212, process 1200 proceeds to block 1208 where the infusion pump performs infusion of the medication as described above. For example, the infusion pump may be configured to control the pump engine to advance medication through an infusion set and into a patient via one or more needles.

An illustrative implementation of a computer system 1800 that may be used in connection with any of the embodiments of the technology described herein is shown in FIG. 18. The computer system 1800 may include one or more processors 810 and one or more articles of manufacture that comprise non-transitory computer-readable storage media (e.g., memory 1820 and one or more non-volatile storage media 1830). The processor 1810 may control writing data to and reading data from the memory 1820 and the non-volatile storage device 1830 in any suitable manner, as the aspects of the technology described herein are not limited in this respect. To perform any of the functionality described herein, the processor 1810 may execute one or more processor-executable instructions stored in one or more non-transitory computer-readable storage media (e.g., the memory 1820), which may serve as non-transitory computer-readable storage media storing processor-executable instructions for execution by the processor 1810.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the technology described herein need not reside on a single computer or processor, but may be distributed in a modular fashion among different computers or processors to implement various aspects of the technology described herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Based on the foregoing disclosure, it should be apparent to one of ordinary skill in the art that the embodiments disclosed herein are not limited to a particular computer system platform, processor, operating system, network, or communication protocol. Also, it should be apparent that the embodiments disclosed herein are not limited to a specific architecture.

It is to be appreciated that embodiments of the methods and apparatuses described herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features described in connection with any one or more embodiments are not intended to be excluded from a similar role in any other embodiments.

The terms “approximately,” “substantially,” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1-20. (canceled)

21. An infusion pump for administering medication to a patient, the infusion pump configured to couple to an infusion set via which the medication is infused into the patient, the infusion pump comprising:
a processor; and
at least one non-transitory computer-readable storage medium storing processor-executable instructions that, when executed by the processor, cause the processor to:
determine whether the infusion set is configured to infuse the medication into the patient at one site or at two sites;
if it is determined that the infusion set is configured to infuse the medication at one site:
determine a first length of time for infusion of the medication; and
control a pump engine to infuse the medication into the patient based on the first length of time, wherein the pump engine is configured to pump the medication through the infusion set;
if it is determined that the infusion set is configured to infuse the medication at two sites:
determine a second length of time for infusion of the medication, wherein the second length of time is different from the first length of time; and
control the pump engine to infuse the medication into the patient based on the second length of time.

22. The infusion pump of claim 21, further comprising the pump engine.

23. The infusion pump of claim 21, further comprising a sensor, wherein the instructions further cause the processor to determine whether the infusion set is configured to infuse the medication at one site or at two sites at least in part by using the sensor.

24. The infusion pump of claim 21, wherein the instructions further cause the processor to:
determine at least one first maximum dosage of the medication that the patient can be infused with when it is determined that the infusion set is configured to infuse the medication at one site; and
determine at least one second maximum dosage of the medication that the patient can be infused with when it is determined that the infusion set is configured to infuse the medication at two sites, wherein the at least one second maximum dosage is different from the at least one first maximum dosage.

25. The infusion pump of claim 24, wherein the instructions cause the processor to determine the at least one second maximum dosage to be twice the at least one first maximum dosage.

26. The infusion pump of claim 21, wherein the instructions further cause the processor to determine at least one rate at which to infuse the medication in units of mL/hr/site.

27. The infusion pump of claim 21, wherein the infusion pump is further configured to couple to a first type of infusion set for infusing the medication at one site, and a second type of infusion set for infusing the medication at two sites.

28. The infusion pump of claim 27, wherein the instructions further cause the processor to:
determine that the infusion set is configured to infuse the medication at one site based on a determination that the infusion is coupled to the first type of infusion set; and
determine that the infusion set is configured to infuse the medication at two sites based on a determination that the infusion pump is coupled to the second type of infusion set.

29. The infusion pump of claim 27, further comprising a sensor configured to detect a presence of the first type of infusion set and a presence of the second type of infusion set, wherein the instructions further cause the processor to determine whether the infusion set is configured to infuse the medication at one site or at two sites based on a type of infusion set detected by the sensor.

30. The infusion pump of claim 29, further comprising a display, wherein the instructions further cause the processor to:
receive a user input specifying that one of the first type of infusion set or the second type of infusion set is to be used for infusion of the medication;
determine that a detected type of infusion set is different from the type specified by the user input; and
generate a screen on the display indicating that a different type of infusion set was detected than the one specified by the user input.

31. The infusion pump of claim 30, wherein the instructions further cause the processor to generate a screen on the display instructing to load the type of infusion set specified by the user when it is determined that the type of infusion set detected by the sensor is different from the type specified by the user input.

32. A method of administering medication to a patient by an infusion pump, the method comprising:
with a processor:
determining whether an infusion set coupled to the infusion pump is configured to infuse the medication into the patient at one site or at two sites, wherein the infusion pump is configured to infuse the medication into the patient via the infusion set;
if it is determined that the infusion set is configured to infuse the medication at one site:
determining a first length of time for infusion of the medication; and
controlling a pump engine to infuse the medication into the patient based on the first length of time, wherein the pump engine is configured to pump the medication through the infusion set;
if it is determined that the infusion set is configured to infuse the medication at two sites:
determining a second length of time for infusion of the medication, wherein the second length of time is different from the first length of time; and
controlling the pump engine to infuse the medication into the patient based on the second length of time.

33. The method of claim 32, further comprising determining whether the infusion set is configured to infuse the medication at one site or at two sites at least in part by using a sensor.

34. The method of claim 32, further comprising:
determining at least one first maximum dosage of the medication that the patient can be infused with when it is determined that the infusion set is configured to infuse the medication at one site; and
determining at least one second maximum dosage of the medication that the patient can be infused with when it is determined that the infusion set is configured to infuse the medication at two sites, wherein the at least one second maximum dosage is different from the at least one first maximum dosage.

35-37. (canceled)

38. At least one non-transitory computer-readable storage medium storing instructions that, when executed by a processor, cause the processor to perform a method comprising:
determining whether an infusion set coupled to an infusion pump is configured to infuse medication into a patient at one site or at two sites, wherein the infusion pump is configured to infuse the medication into the patient via the infusion set;
if it is determined that the infusion set is configured to infuse the medication at one site:
determining a first length of time for infusion of the medication; and
controlling a pump engine to infuse the medication into the patient based on the first length of time, wherein the pump engine is configured to pump the medication through the infusion set;
if it is determined that the infusion set is configured to infuse the medication at two sites:
determining a second length of time for infusion of the medication, wherein the second length of time is different from the first length of time; and
controlling the pump engine to infuse the medication into the patient based on the second length of time.

39-49. (canceled)

50. A method of administering medication to a patient by an infusion pump, the method comprising:
with a processor:
generating one or more screens on a display of the infusion pump prompting for user input to prime an infusion set coupled to the infusion pump, the infusion pump configured to infuse the medication into the patient via the infusion set;
receiving one or more user inputs for priming the infusion set for infusing the medication into the patient; and
in response to receiving the one or more user inputs, controlling a pump engine to cause an amount of the medication to advance from a source container through the infusion set, wherein the pump engine is configured to pump the amount of the medication from the source container through the infusion set.

51-55. (canceled)

56. At least one non-transitory computer-readable storage medium storing instructions that, when executed by a processor, cause the processor to perform a method comprising:
generating one or more screens on a display of an infusion pump prompting for user input to prime an infusion set coupled to the infusion pump, wherein the infusion pump is configured to infuse medication into a patient via the infusion set;
receiving one or more user inputs for priming the infusion set for infusing medication into patient; and
in response to receiving the one or more user inputs, controlling a pump engine to cause an amount of the medication to advance from a source container through the infusion set, wherein the pump engine is configured to pump the amount of the medication from the source container through the infusion set.

57-60. (canceled)

61. An infusion pump for administering medication to a patient, the infusion pump comprising:
a processor; and
at least one non-transitory computer-readable storage medium storing processor-executable instructions that, when executed by the processor, cause the processor to:
control a pump engine to pump the medication from a source container into the patient, the pump engine being configured to couple to and fluidly communicate with an infusion set comprising:
an inlet configured to receive medication from the source container; and
an outlet configured to deliver the medication to the patient;
wherein the pump engine is operable in a delivery mode in which fluid is drawn from the source container through the inlet toward the outlet, and in a reverse mode in which fluid is drawn through the outlet toward the inlet;
receive a user input to initiate a site check for determining whether a needle entered a blood vessel of the patient; and
in response to receiving the user input, control the pump engine to operate in the reverse mode to draw fluid from the patient in a direction moving from the outlet toward the inlet.

62-66. (canceled)

67. A method of administering medication to a patient by an infusion pump, the method comprising:
with a processor:
controlling a pump engine to pump the medication from a source container into the patient, the pump engine being configured to couple to and fluidly communicate with an infusion set comprising:
an inlet configured to receive medication from the source container; and
an outlet configured to deliver the medication to the patient;
wherein the pump engine is operable in a delivery mode in which fluid is drawn from the source container through the inlet toward the outlet, and in a reverse mode in which fluid is drawn through the outlet toward the inlet;
receiving a user input to initiate a site check for determining whether needle entered a blood vessel of the patient; and
in response to receiving the user input, controlling the pump engine to operate in the reverse mode to draw fluid from the patient in a direction moving from the outlet toward the inlet.

68-71. (canceled)

72. At least one non-transitory computer-readable storage medium storing instructions that, when executed by a processor, cause the processor to perform a method comprising:
controlling a pump engine to pump a medication from a source container into a patient, the pump engine being configured to couple to and fluidly communicate with an infusion set comprising:
an inlet configured to receive medication from the source container; and
an outlet configured to deliver medication to the patient;
wherein the pump engine is operable in a delivery mode in which fluid is drawn from the source container through the inlet toward the outlet, and in a reverse mode in which fluid is drawn through the outlet toward the inlet;
receiving a user input to initiate a site check for determining whether a needle entered a blood vessel of the patient; and
in response to receiving the user input, controlling the pump engine to operate in the reverse mode to draw fluid from the patient in a direction moving from the outlet toward the inlet.

73-76. (canceled)
